(19) United States
(12) Patent Application Publication
(10) Pub. No.: US 2024/0217919 A1
(43) Pub. Date: Jul. 4, 2024

(54) NOVEL METHODS FOR SYNTHESIZING ENANTIOPURE (S)-METHADONE, (R)-METHADONE, RACEMIC (R,S)-METHADONE AND RELATED ANALOGUE SUBSTANCES

(71) Applicants: Paolo L. Manfredi, Miami Beach, FL (US); Charles E. Inturrisi, New York, NY (US)

(72) Inventors: Paolo L. Manfredi, Miami Beach, FL (US); Charles E. Inturrisi, New York, NY (US); Andrea Mattarei, Perarolo di Vigonza (IT); Marco Banzato, Galzignano Terme (IT); Alberto Furlan, Padua (IT); Alberto Ongaro, Campolongo Maggiore (IT); Brian H. Heasley, Wake Forest, NC (US); Zachary D. Tucker, Cary, NC (US)

(21) Appl. No.: 18/554,025

(22) PCT Filed: Apr. 8, 2022
(86) PCT No.: PCT/US2022/023950

§ 371 Date: Oct. 5, 2023

Related U.S. Application Data
(60) Provisional application No. 63/172,901, filed on Apr. 9, 2021.

(51) Int. Cl. C07C 221/00; C07C 225/16

(52) U.S. Cl. CPC C07C 221/00; C07C 225/16

(57) ABSTRACT

Methods for the synthesis of enantiopure (S)-methadone, (R)-methadone, (R,S)-methadone and related analogues starting from optically active or racemic N-substituted cyclic sulfamidates, or N-substituted aziridines with retention of stereochemical configuration. The methods avoid the formation of isomethadone-nitrile by-products observed under the previously reported conditions, namely the ring-opening reaction of the corresponding 1,1,2-trimethylaziridin-1-ium salt and the 2,2-diphenylacetonitrile anion, resulting in improved yields and facile purification operations.

### CROSS-REFERENCE TO RELATED APPLICATION

This application claims priority to, and the benefit of the filing date of, U.S. Patent Application Ser. No. 63/172,901, filed on Apr. 9, 2021, the disclosure of which is incorporated by reference herein in its entirety.

### FIELD OF THE INVENTION

The present invention relates generally to methods for the synthesis of enantiopure (S)-methadone, (R)-methadone, (R,S)-methadone, and related analogues.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

As initially discovered by the applicants, (S)-methadone, the dextrorotatory isomer of methadone (which may also be referred to herein as dextromethadone, esmethadone, and/or REL-1017) is an uncompetitive, low-affinity, low-potency N-methyl-D-aspartate (NMDA) receptor antagonist displaying a preference for pathologically and tonically open, hyperactive ion channels, thus prompting potential effectiveness for a multiplicity of diseases and disorders at well tolerated dosages, without psychotomimetic effects (Gorman A L, Elliott K J, Inturrisi C E. The d- and l-isomers of methadone bind to the non-competitive site on the N-methyl-D-aspartate (NMDA) receptor in ratforebrain and spinal cord. Neurosci Lett. 1997 Feb. 14; 223(1):5-8. doi: 10.1016/s0304-3940(97)13391-2. PMID: 9058409; Bernstein G, Davis K, Mills C, Wang L, McDonnell M, Oldenhof J, Inturrisi C, Manfredi P L, Vitolo O V. Characterization of the Safety and Pharmacokinetic Profile of D-Methadone, a Novel N-Methyl-D-Aspartate Receptor Antagonist in Healthy, Opioid-Naive Subjects: Results of Two Phase 1 Studies. J Clin Psychopharmacol. 2019 May/June; 39(3):226-237. doi: 10.1097/JCP.0000000000001035. PMID: 30939592; Fava M, Stahl S, Pani L, De Martin S, Pappagallo M, Guidetti C, Alimonti A, Bettini E, Mangano R M, Wessel T, de Somer M, Caron J, Vitolo O V, DiGuglielmo G R, Gilbert A, Mehta H, Kearney M, Mattarei A, Gentilucci M, Folli F, Traversa S, Inturrisi C E, Manfredi P L. REL-1017 (Esmethadone) as Adjunctive Treatment in Patients With Major Depressive Disorder: A Phase 2a Randomized Double-Blind Trial. Am J Psychiatry. 2022 February; 179(2):122-131. doi: 10.1176/appi.ajp.2021.21020197. Epub 2021 Dec. 22. PMID: 34933568). Esmethadone exhibits a 20-fold lower affinity for the opioid receptors compared to the enantiomeric (R)-methadone (Codd E E, Shank R P, Schupsky J J, Raffa R B. Serotonin and norepinephrine uptake inhibiting activity of centrally acting analgesics: structural determinants and role in antinociception. J Pharmacol Exp Ther. 1995 September; 274(3):1263-70. PMID: 7562497), and does not display meaningful opioid agonist effects, including addiction liability.

Racemic methadone is used for the treatment of opioid use disorder (OUD) and for the treatment of pain. Based on clinical and experimental evidence, half dose of (R)-methadone instead of (R,S)-methadone can be administered to patients (Gutwinski S, Schoofs N, Stuke H, Riemer T G, Wiers C E, Bermpohl F. Opioid tolerance in methadone maintenance treatment: comparison of methadone and levomethadone in long-term treatment. Harm Reduct J. 2016 Feb. 16; 13:7. doi: 10.1186/s12954-016-0095-0. PMID: 26879120; PMCID: PMC4754801; Soyka M, Zingg C. Feasability and safety of transfer from racemic methadone to (R)-methadone in primary care: clinical results from an open study. World J Biol Psychiatry. 2009; 10(3):217-24. doi: 10.1080/15622970802416057. PMID: 19629858). The administration of the (R)-form would decrease plasma concentrations of methadone by two-fold, thereby producing a safer safety profile, while not affecting the activity on the mu opioid receptors. Prescription of (R)-methadone would also diminish the clinical concern of CYP2B6 slow metabolizer status for cardiotoxic effects. At the doses used to treat OUD and pain, (R)-methadone may therefore confer a lower risk of QTc interval prolongation (time from the start of the Q wave to the end of the T wave, time taken for ventricular depolarisation and repolarization) resulting in a better safety profile compared to (R,S)-methadone (Grilo L S, Carrupt P A, Abriel H. Stereoselective Inhibition of the hERGI Potassium Channel. Front Pharmacol. 2010 Nov. 22; 1:137. doi: 10.3389/fphar.2010.00137. PMID: 21833176; PMCID: PMC3153011).

The emerging importance of the two methadone isomers and the benefits of their administration as separate drugs (rather than the administration of racemic methadone), underscores the importance of developing cost-effective procedures for the synthesis of methadone isomers with high optical purity.

Beckett et al. reported the first protocol for a stereoselective synthesis of (R)-methadone starting from (R)-alanine in 1957 [Beckett, A. H., and N. J. Harper. “162. Configurational studies in synthetic analgesics: the synthesis of (−)-methadone from D-(−)-alanine.” Journal of the Chemical Society (Resumed) (1957): 858-861]. This protocol included a seven step procedure resulting in roughly 5% overall yield with an optical purity of approximately 72% for the final levomethadone. Such a procedure, in principle, could be utilized for the preparation of both (R)-, and (S)-methadone. However, the complex synthetic route, low optical purity, and poor overall yield are not practical and discourage its use, especially for exploitation in the pharmaceutical industry.

In 2000, U.S. Pat. No. 6,143,933 by Scheinmann et al. provided a method involving the enzymatic resolution of the racemic intermediate 1-(dimethylamino)propan-2-ol followed by conversion of the enantiomers to optically active (R)- and (S)-methadone with about 3% overall yield and enantiomeric excess greater than 99%. Despite the excellent enantiomeric excess achieved, the overall yield for this process is prohibitively low and the involvement of an enzymatic reaction is not practical and contemplates a high-cost incurrence for industrial-scale processes. Additionally, methods based on the racemic synthesis of methadone followed by resolution of enantiomers (including those disclosed in U.S. Publication No. US2014/0350302) suffer from the introduction of undesirable impurities, complex operations, and significant reduction in the overall yield of the desired enantiomer, resulting in strategies for large scale manufacturing that are not appealing.

U.S. Publication No. 2017/0057909 by Mkrtchyan et al. represented an advance in the state of the art for the chiral synthesis of either (R)- or (S)-methadone providing more efficient methods for the synthesis of optically active methadones starting from (R)- or (S)-alanine respectively with about 20% overall yield and enantiomeric excess greater than 99%. The best performing enantioselective procedure provided by Mkrtchyan et al. for the synthesis of optically pure methadones consists of converting (R)- or (S)-alanine to the corresponding enantiopure N,N-dimethyl-alanine; reduction of the N,N-dimethyl-alanine to form N,N-dimethyl-alaninol; converting the N,N-dimethyl-alaninol to the corresponding chloride intermediate via a substitution reaction; mixing the chloride intermediate and a base, to form 1,1,2-trimethylaziridin-1-ium in situ, then treatment with 2,2-diphenylacetonitrile to provide enantiopure methadone-nitrile; and finally exposing the methadone-nitrile to ethyl magnesium bromide to form an ethyl imine intermediate, which is subsequently hydrolyzed in the presence of hydrochloric acid to provide optically pure methadone hydrochloride (See FIG. 1, which depicts this synthesis strategy with respect to (S)-methadone).

When one looks to the best results among the two enantiomers in the examples provided in Mkrtchyan's US 2017/0057909 using this synthetic strategy, the reagents and conditions chosen (and resulting yields) are: i) CH₂O, Pd/C, H₂, water, 50° C. for 20 h, then reflux for 1 h, (85% yield); ii) LiAlH₄, THF, 0° C. to reflux, 16 h, (68% yield); iii) SOCl₂, CHCl₃, 0° C. to reflux, 1 h, (93% yield); iv) KOtBu, Ph₂CHCN, DMF, rt for 2.5 h and then 45° C. for 15 h, (38% yield); v) EtMgBr, toluene, 110° C. for 3 h, then 6M HCl, 75° C. for 3 h, (88% yield). Overall yield: 18%

All the synthetic procedures disclosed in the literature (for example WO2013/168000, WO2017/35224, WO2013/77720, and US2015/152081) are based on one key step shown at step “iv” in FIG. 1, namely the attack of 2,2-diphenylacetonitrile anion on the 1,1,2-trimethylaziridin-1-ium generated by an intramolecular S_{N}2-reaction of (generally) 1-dimethylamino-2-chloropropane under basic conditions. The diphenylacetonitrile anion can then attack from two sides of the aziridinium salt resulting in competitive ring-opening reactions with low levels of regiochemical discrimination. In case of an attack at the least substituted C-atom of the aziridinium salt, the desired methadone-nitrile is formed. However, if the attack occurs at the higher order C-atom, the by-product isomethadone-nitrile is formed (See FIG. 2). Under the optimal mixture of base, solvent, and reaction conditions, the outcome of this reaction is at best a 1:3 (isomethadone-nitrile:methadone-nitrile) mixture of the two regioisomeric products. The resulting mixture is then purified by adopting a complex crystallization process that exploit the higher solubility isomethadone-nitrile, leading to non-efficient recovery and low purified yield of the desired methadone-nitrile.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

As described above, the emerging importance of the two methadone isomers, and the benefits of their administration as separate drugs, underscores the importance of developing cost-effective procedures for the synthesis of methadone isomers with high optical purity. And so, one aspect of the present invention is directed to novel enantioselective approaches for the synthesis of (S)-methadone, (R)-methadone, (R,S)-methadone and their analogues. In particular, one aspect includes a method for preparing (S)-methadone, (R)-methadone or (R,S)-methadone from optically pure ((S)- or (R)-) or racemic N-protected 4-methyl-cyclic sulfamidate or N-protected 2-methylaziridine. In certain embodiments, the process includes a ring opening reaction of optically pure ((S)- or (R)-) or racemic N-protected 4-methyl-cyclic sulfamidate or N-protected 2-methylaziridine with 2,2-diphenylacetonitrile in the presence of a base to provide ((S)- or (R)-) or racemic N-protected-dinormethadone nitrile with retention of configuration. This ring opening reaction is then followed by a two-step/one-pot deprotection/reductive amination of the N-protected-dinormethadone nitrile to provide (R)-, (S)-, or (R,S)-methadone nitrile. A reaction of the (R)-, (S)-, or (R,S)-methadone nitrile with an organomagnesium halide reagent to form an ethyl-imine intermediate is then performed—followed by imine hydrolysis to provide (R)-, (S)-, or (R,S)-methadone.

Another aspect of the present invention is directed to a method for preparing (S)-methadone hydrochloride, (R)-methadone hydrochloride or (R,S)-methadone hydrochloride from optically pure ((S)- or (R)-) or racemic N-protected 4-methyl-cyclic sulfamidate or N-protected 2-methylaziridine. In certain embodiments, the process includes a ring opening reaction of optically pure ((S)- or (R)-) or racemic N-protected 4-methyl-cyclic sulfamidate or N-protected 2-methylaziridine with diphenylacetonitrile in the presence of a base to provide ((S)- or (R)-) or racemic N-protected-dinormethadone nitrile with retention of configuration. This ring opening reaction is then followed by a two-step/one-pot deprotection/reductive amination of the N-protected-dinormethadone nitrile to provide (R)-, (S)-, or (R,S)-methadone nitrile. A reaction of the (R)-, (S)-, or (R,S)-methadone nitrile with an organomagnesium halide reagent to form an ethyl-imine intermediate is then performed—followed by hydrochloric acid-mediated imine hydrolysis to provide (R)-, (S)-, or (R,S)-methadone hydrochloride.

Another aspect of the present invention is directed to a compound (or pharmaceutically acceptable salt thereof, prepared by the method of the two aspects described above. In various embodiments, the compound (or pharmaceutically acceptable salt thereof) has the formula:

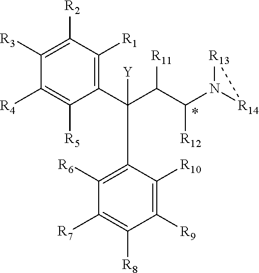

wherein (1) R₁ to R₁₀ are, independently for each occurrence, hydrogen, deuterium, halogen, alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate; (2) R₁ and R₁₂ are, independently for each occurrence, hydrogen, deuterium, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate; or R₁ and R₁₂ are, independently for each occurrence, selected from the group consisting of halogen, alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate; (3) NR₁₃R₁₄ is optionally cyclized through C₃-C₁₂ cycloalkyl, or heterocyclyl, any of which are optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate; (4) if NR₁₃R₁₄ is not cyclized R₁₃ and R₁₄ are, independently for each occurrence, hydrogen, deuterium, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate, or R₁₃ and R₁₄ are, independently for each occurrence, selected from the group consisting of alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate; (5) Y is cyano, —C(O)R₁₅, —C(OH)R₁₅, —C(OR₁₆)R₁₅. R₁₅ and R₁₆ are, independently for each occurrence, hydrogen, deuterium, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate; or R₁₅ and R₁₆ are, independently for each occurrence, selected from the group consisting of alkyl ester, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate; and (6) * is a stereocenter selected from (R)-, (S)- or (R,S)-configuration, wherein the stereocenter is in an R configuration, and wherein the compound is present in greater than 90%, 95%, 97%, 99%, 99.5%, or 99.9% enantiomeric excess (e.e) compared to the S isomer, or wherein the stereocenter is in an S configuration, and wherein the compound is present in greater than 90%, 95%, 97%, 99%, 99.5%, or 99.9% enantiomeric excess (e.e.) compared to the R isomer.

A further aspect of the present invention is directed to a method for the chromatography free and scalable preparation of a cyclic sulfamidate starting material selected from benzyl (S)-4-methyl-1,2,3-oxathiazolidine-3-carboxylate 2,2-dioxide, (R)-4-methyl-1,2,3-oxathiazolidine-3-carboxylate 2,2-dioxide, and (R,S)-4-methyl-1,2,3-oxathiazolidine-3-carboxylate 2,2-dioxide.

A further aspect of the present invention is directed to a method for preparing (R)-, (S)-, or (R,S)-methadone nitrile. In this aspect, the method includes a ring opening reaction of optically pure ((S)- or (R)-) or racemic N-protected 4-methyl-cyclic sulfamidate or N-protected 2-methylaziridine with diphenylacetonitrile in the presence of a base to provide ((S)- or (R)-) or racemic N-protected-dinormethadone nitrile with retention of configuration. And this step is then followed by a two-step/one-pot deprotection/reductive amination of the N-protected-dinormethadone nitrile to provide (R)-, (S)-, or (R,S)-methadone nitrile.

Another aspect of the present invention is directed to a ring opening reaction of optically pure ((S)- or (R)-) or racemic N-protected 4-methyl-cyclic sulfamidate or N-protected 2-methylaziridine with diphenylacetonitrile in the presence of a base to provide ((S)- or (R)-) or racemic N-protected-dinormethadone nitrile with retention of configuration.

### BRIEF DESCRIPTION OF THE DRAWINGS

The accompanying drawings, which are incorporated in and constitute a part of this specification, illustrate embodiments of the invention and, together with the general description of the invention given above and the detailed description of the embodiments given below, serve to explain the principles of the present invention.

FIG. 1 is a schematic showing the synthesis of (S)-methadone using the strategy proposed in U.S. Publication No. 2017/0057909.

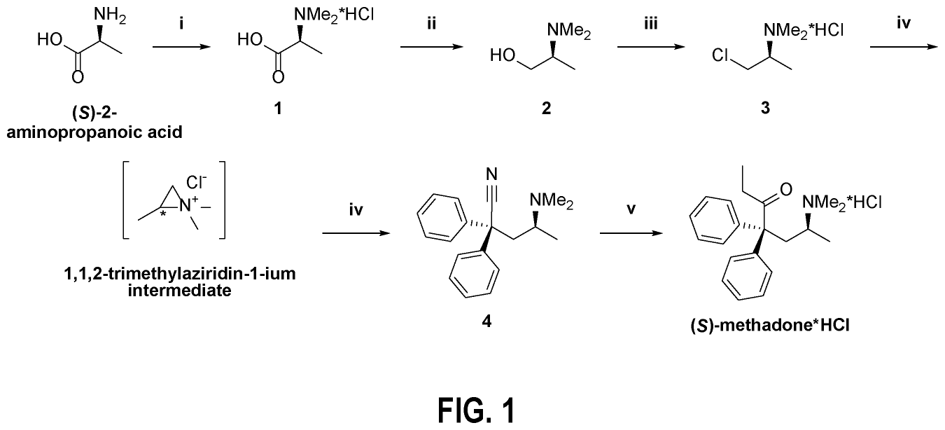

FIG. 2 is a schematic showing a key step in previously reported procedures for the synthesis of (R)-, (S)- and (R,S)-methadone.

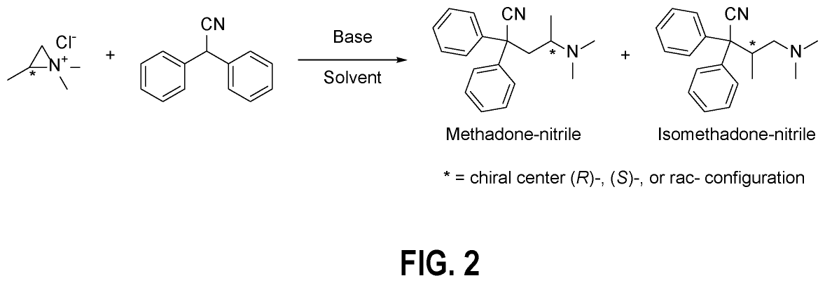

FIG. 3 is a schematic representation of a procedure for preparation of (R)-, (S)- or, (R,S)-methadone.

FIG. 4 is a schematic representation of a procedure for preparation of N-protected cyclic sulfamidate starting material.

FIG. 5 is a schematic representation of the preparation of N-protected 2-methylaziridine starting material.

FIG. 6 shows the general structure of enantiopure or racemic methadone analogues that can be synthesized using the disclosed methods.

FIG. 7 is a schematic showing the synthesis of Cbz-protected (S)-cyclic sulfamidate starting material.

FIG. 8 is a graph showing the ¹H NMR spectrum of benzyl (S)-(1-hydroxypropan-2-yl)carbamate.

FIG. 9 is a graph showing the ¹H NMR spectrum of benzyl (S)-4-methyl-1,2,3-oxathiazolidine-3-carboxylate 2,2-dioxide.

FIG. 10 is a graph showing the ¹³C NMR spectrum of benzyl (S)-4-methyl-1,2,3-oxathiazolidine-3-carboxylate 2,2-dioxide.

FIG. 11 is a schematic showing the synthesis of Boc-protected (R)-aziridine starting material.

FIG. 12 is a graph showing the ¹H NMR spectrum of tert-butyl (R)-(1-hydroxypropan-2-yl)carbamate.

FIG. 13 is a graph showing the ¹³C NMR spectrum of tert-butyl (R)-(1-hydroxypropan-2-yl)carbamate.

FIG. 14 is a graph showing the ¹H NMR spectrum of tert-butyl (R)-2-methylaziridine-1-carboxylate.

FIG. 15 is a graph showing the ¹³C NMR spectrum of tert-butyl (R)-2-methylaziridine-1-carboxylate.

FIG. 16 is a schematic showing the synthesis of (S)-methadone nitrile using Cbz-protected (S)-cyclic sulfamidate starting material.

FIG. 17 is a graph showing the ¹H NMR spectrum of benzyl (S)-(4-cyano-4,4-diphenylbutan-2-yl)carbamate.

FIG. 18 is a graph showing the ¹³C NMR spectrum of benzyl (S)-(4-cyano-4,4-diphenylbutan-2-yl)carbamate.

FIG. 19A is a graph and table showing a Chiral HPLC chromatogram of (R,S)-methadone nitrile.

FIG. 19B is a graph and table showing a Chiral HPLC chromatogram of (S)-methadone nitrile.

FIG. 20 is a graph showing the ¹H NMR spectrum of (S)-methadone nitrile.

FIG. 21 is a graph showing the ¹³C NMR spectrum of (S)-methadone nitrile.

FIG. 22 is a schematic showing the synthesis of (R)-methadone nitrile using Boc-protected (R)-2-methylaziridine starting material.

FIG. 23 is a graph showing the ¹H NMR spectrum of tert-butyl (R)-(4-cyano-4,4-diphenylbutan-2-yl)carbamate.

FIG. 24 is a graph showing the ¹³C NMR spectrum of tert-butyl (R)-(4-cyano-4,4-diphenylbutan-2-yl)carbamate.

FIG. 25A is a chiral HPLC chromatogram of (R,S)-methadone nitrile.

FIG. 25B is a chiral HPLC chromatogram of (R)-methadone nitrile.

FIG. 26 is a graph showing the ¹H NMR spectrum of (R)-methadone nitrile.

FIG. 27 is a graph showing the ¹³C NMR spectrum of (R)-methadone nitrile.

FIG. 28 is a schematic showing the synthesis of (S)-methadone hydrochloride from (S)-4-(dimethylamino)-2,2-diphenylpentanenitrile.

FIG. 29A is a chiral HPLC chromatogram of (R,S)-methadone.

FIG. 29B is a chiral HPLC chromatogram of (S)-methadone.

FIG. 30 is a graph showing the ¹H NMR spectrum of (S)-methadone*HCl.

FIG. 31 is a graph showing the ¹³C NMR spectrum of (S)-methadone*HCl.

FIG. 32 is a schematic showing the synthesis of (R)-methadone hydrochloride from (R)-4-(dimethylamino)-2,2-diphenylpentanenitrile.

FIG. 33A is a chiral HPLC chromatogram of (R,S)-methadone.

FIG. 33B is a chiral HPLC chromatograms of (R)-methadone.

FIG. 34 is a graph showing the ¹H NMR spectrum of (R)-methadone*HCl.

FIG. 35 is a graph showing the ¹³C NMR spectrum of (R)-methadone*HCl.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As described above, one aspect of the present invention is directed to a method for preparing (S)-methadone, (R)-methadone or (R,S)-methadone from optically pure ((S)- or (R)-) or racemic N-protected 4-methyl-cyclic sulfamidate or N-protected 2-methylaziridine. In certain embodiments, the process includes a ring opening reaction of optically pure ((S)- or (R)-) or racemic N-protected 4-methyl-cyclic sulfamidate or N-protected 2-methylaziridine with 2,2-diphenylacetonitrile in the presence of a base to provide ((S)- or (R)-) or racemic N-protected-dinormethadone nitrile with retention of configuration. This ring opening reaction is then followed by a two-step/one-pot deprotection/reductive amination of the N-protected-dinormethadone nitrile to provide (R)-, (S)-, or (R,S)-methadone nitrile. A reaction of the (R)-, (S)-, or (R,S)-methadone nitrile with an organomagnesium halide reagent to form an ethyl-imine intermediate is then performed—followed by imine hydrolysis to provide (R)-, (S)-, or (R,S)-methadone.

Another aspect of the present invention is directed to a method for preparing (S)-methadone hydrochloride, (R)-methadone hydrochloride or (R,S)-methadone hydrochloride from optically pure ((S)- or (R)-) or racemic N-protected 4-methyl-cyclic sulfamidate or N-protected 2-methylaziridine. In certain embodiments, the process includes a ring opening reaction of optically pure ((S)- or (R)-) or racemic N-protected 4-methyl-cyclic sulfamidate or N-protected 2-methylaziridine with diphenylacetonitrile in the presence of a base to provide ((S)- or (R)-) or racemic N-protected-dinormethadone nitrile with retention of configuration. This ring opening reaction is then followed by a two-step/one-pot deprotection/reductive amination of the N-protected-dinormethadone nitrile to provide (R)-, (S)-, or (R,S)-methadone nitrile. A reaction of the (R)-, (S)-, or (R,S)-methadone nitrile with an organomagnesium halide reagent to form an ethyl-imine intermediate is then performed—followed by hydrochloric acid-mediated imine hydrolysis to provide (R)-, (S)-, or (R,S)-methadone hydrochloride.

Another aspect of the present invention is directed to a compound (or pharmaceutically acceptable salt thereof, prepared by the method of the two aspects described above. In various embodiments, the compound (or pharmaceutically acceptable salt thereof) has the formula:

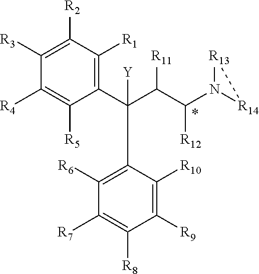

wherein (1) R₁ to R₁₀ are, independently for each occurrence, hydrogen, deuterium, halogen, alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate; (2) R₁₁ and R₁₂ are, independently for each occurrence, hydrogen, deuterium, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate; or Ru and R₁₂ are, independently for each occurrence, selected from the group consisting of halogen, alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate; (3) NR₁₃R₁₄ is optionally cyclized through C₃-C₁₂ cycloalkyl, or heterocyclyl, any of which are optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate; (4) if NR₁₃R₁₄ is not cyclized R₁₃ and R₁₄ are, independently for each occurrence, hydrogen, deuterium, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate, or R₁₃ and R₁₄ are, independently for each occurrence, selected from the group consisting of alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate; (5) Y is cyano, —C(O)R₁₅, —C(OH)R₁₅, —C(OR₁₆)R₁₅. R₁₅ and R₁₆ are, independently for each occurrence, hydrogen, deuterium, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate; or R₁₅ and R₁₆ are, independently for each occurrence, selected from the group consisting of alkyl ester, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate; and (6) * is a stereocenter selected from (R)-, (S)- or (R,S)-configuration, wherein the stereocenter is in an R configuration, and wherein the compound is present in greater than 90%, 95%, 97%, 99%, 99.5%, or 99.9% enantiomeric excess (e.e) compared to the S isomer, or wherein the stereocenter is in an S configuration, and wherein the compound is present in greater than 90%, 95%, 97%, 99%, 99.5%, or 99.9% enantiomeric excess (e.e) compared to the R isomer.

A further aspect of the present invention is directed to a method for the chromatography free and scalable preparation of a cyclic sulfamidate starting material selected from benzyl (S)-4-methyl-1,2,3-oxathiazolidine-3-carboxylate 2,2-dioxide, (R)-4-methyl-1,2,3-oxathiazolidine-3-carboxylate 2,2-dioxide, and (R,S)-4-methyl-1,2,3-oxathiazolidine-3-carboxylate 2,2-dioxide.

Yet a further aspect of the present invention is directed to a method for preparing (R)-, (S)-, or (R,S)-methadone nitrile. In this aspect, the method includes a ring opening reaction of optically pure ((S)- or (R)-) or racemic N-protected 4-methyl-cyclic sulfamidate or N-protected 2-methylaziridine with diphenylacetonitrile in the presence of a base to provide ((S)- or (R)-) or racemic N-protected-dinormethadone nitrile with retention of configuration. And this step is then followed by a two-step/one-pot deprotection/reductive amination of the N-protected-dinormethadone nitrile to provide (R)-, (S)-, or (R,S)-methadone nitrile.

And another aspect of the present invention is directed to a ring opening reaction of optically pure ((S)- or (R)-) or racemic N-protected 4-methyl-cyclic sulfamidate or N-protected 2-methylaziridine with diphenylacetonitrile in the presence of a base to provide ((S)- or (R)-) or racemic N-protected-dinormethadone nitrile with retention of configuration.

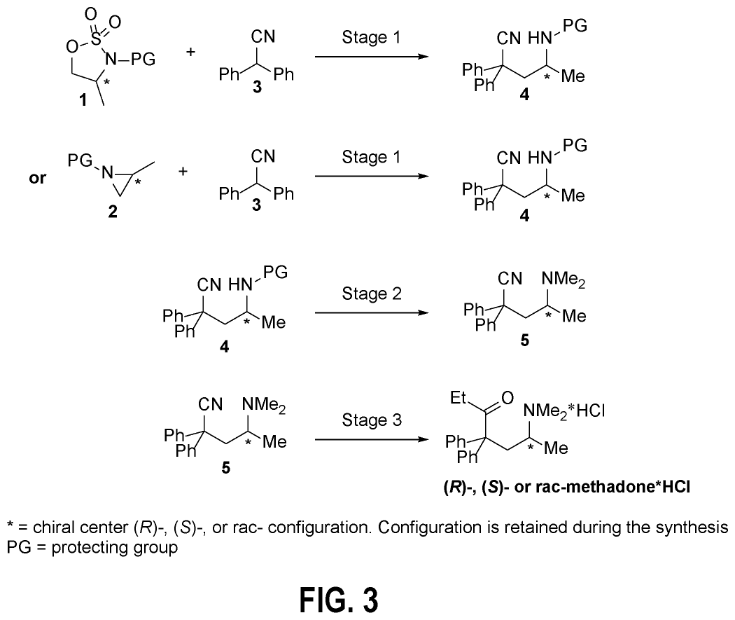

The methods for the synthesis of (S)-methadone, (R)-methadone, (R,S)-methadone and analogues disclosed herein were envisioned and developed by the present inventors as enantio-retentive, scalable, three-stage processes concluding with the isolation of the (R), (S), or (R,S)-methadone in the hydrochloric salt form. Referring to FIG. 3, major features of these methods are the strategic exploitation of the chiral pool employing (R), (S), or (R,S)-alaninol-derived N-protected cyclic 4-methyl-sulfamidate (1), or N-protected 2-methylaziridine (2) as key starting materials of the synthetic process. The N-protecting group can be chosen among the most common carbamate protecting groups for amines [e.g., fluorenylmethyloxycarbonyl (Fmoc), tert-butyloxycarbonyl (Boc), carboxybenzyl (Cbz), acetyl (C(O)CH₃), trifluoroacetyl (C(O)CF₃), benzyl (Bn), triphenylmethyl (CPh₃), p-toluenesulfonyl (Ts)].

Previously reported processes for the synthesis of (R)- and (S)-methadone consist of nucleophilic ring-opening reactions of aziridiniums. However, these technologies suffer from poor regioselectivity, resulting in isomeric impurities complicating purification and isolation of the desired nitrile intermediate (see, e.g., the processes disclosed in US 2020/0277250 A1; WO, 2017/035225 A1; WO 2017/035524 A1; US 2000/6143933; and US 2018/10040752 B2). Furthermore, unlike the ring-opening of N-protected cyclic 4-methyl-sulfamidate (1) or N-protected 2-methylaziridine (2), the aziridinium strategy can result in loss of optical purity, requiring resolution techniques for the isolation of enantiopure material (as in WO 2013/077720 A1; WO 1997/45551 A1; IN 2014/MU04118 A; US 2015/9212128 B2).

In the disclosed methods in accordance with aspects of the present invention—and still referring to FIG. 3—Stage 1 of the present method was useful for the minimization of regioisomeric by-products observed in previous synthesis (i.e. isomethadone-nitrile). Employing N-protected cyclic 4-methyl-sulfamidate (1) as starting material for the reaction with 2,2-diphenylacetonitrile (3) in the presence of a base, resulted in excellent yield and purity of the N-protected intermediate (4) following crystallization. Similarly, reaction of N-protected 2-methylaziridine (2) with 2,2-diphenylacetonitrile (3) in the presence of a base, resulted in good yield and efficient conversion to the N-protected intermediate (4) after purification.

Stage 2 of the present method (as noted above) is a two-step/one-pot deprotection/reductive amination of the N-protected intermediate (4), resulting in the synthesis of common intermediate (R)-, (S)-, or (R,S)-methadone nitrile (5), in good yield and purity. Reaction conditions of Stage 2 depend on the N-protecting group adopted, as shown in the Examples provided below.

In Stage 3 of the present method (as noted above), treatment of the intermediate (R)-, (S)-, or (R,S)-methadone nitrile (5) with a slight excess of EtMgBr (one example of a organomagnesium halide agent) resulted in the generation of the corresponding ethyl-imine, which was hydrolyzed under acidic conditions to provide (R)-, (S)-, or (R,S)-methadone as already disclosed. The desired (R)-, (S)-, or (R,S)-methadone was isolated via a seeded crystallization in good yield and high purity.

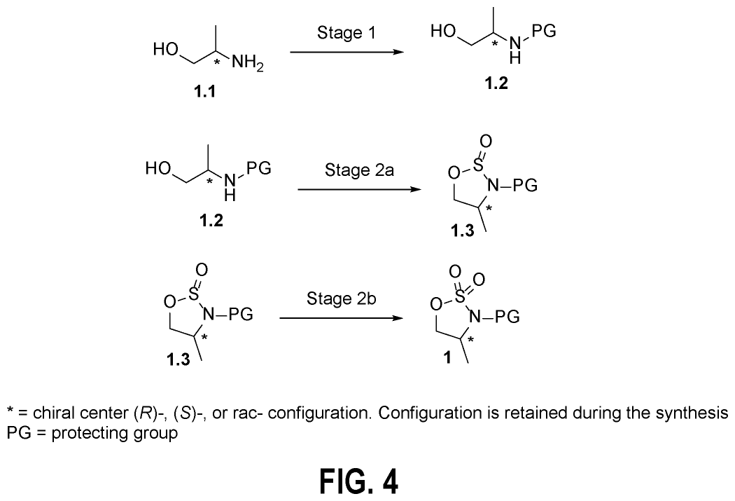

Referring now to FIG. 4, N-protected cyclic 4-methyl-sulfamidate (one possible starting material) can be produced in a two-stage process starting from (R)-, (S)-, or (R,S)-2-aminopropan-1-ol (1.1). The first stage is the protection of the amino group with the selected protecting group to obtain the corresponding N-protected (R)-, (S)-, or (R,S)-2-aminopropan-1-ol (1.2). The second stage is a two-steps/one-pot process to obtain first the N-protected cyclic sulfamidite intermediate (1.3 in Stage 2a) by cyclization reaction with thionyl chloride in the presence of a base followed by the oxidation of the sulfur atom in the presence of sodium periodate as an oxidizing agent and ruthenium chloride as a catalyst (Stage 2b) to obtain the desired N-protected cyclic sulfamidate (1) in good yield and purity after crystallization (See FIG. 4).

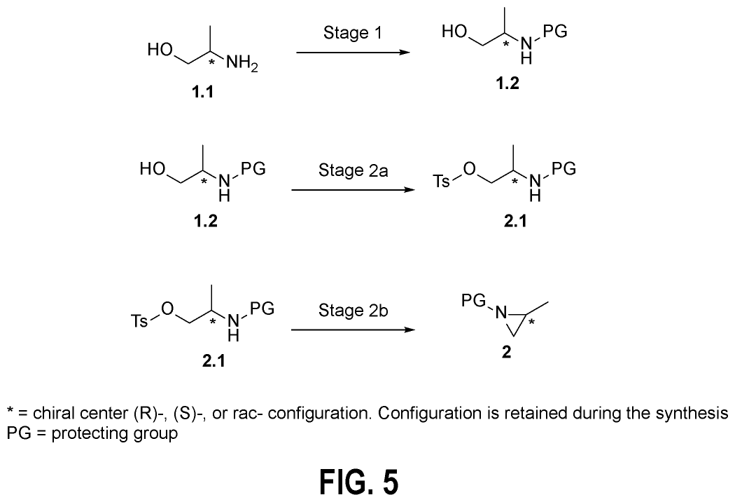

N-protected 2-methylaziridine (an alternate starting material) can be produced in a two-stage process as already disclosed for example in WO 2012/014109 or WO2019/52545 starting from (R)-, (S)-, or (R,S)-2-aminopropan-1-ol (1.1). Referring to FIG. 5, the first stage in this production is the protection of the amino group with the selected carbamate protecting group to obtain the corresponding N-protected (R)-, (S)-, or (R,S)-2-aminopropan-1-ol (1.2). The second stage is a two-step/one-pot process to obtain initially the N-protected (R)-, (S)-, or (R,S)-2-aminopropan-1-tosylate intermediate (2.1 in Stage 2a) by reaction with p-toluenesulfonyl chloride in the presence of a base followed by aziridine ring closure (Stage 2b) to obtain the desired N-protected 2-methylaziridine (2) in good yield and purity.

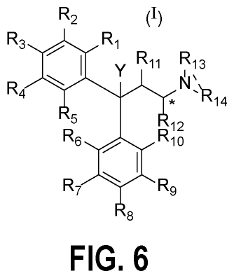

As mentioned above, the methods disclosed herein are also applicable to the synthesis of methadone analogues according to the general Formula (I)—shown below and also in FIG. 6:

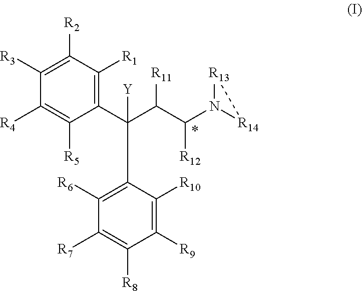

wherein:
R₁ to R₁₀ are, independently for each occurrence, hydrogen, deuterium, halogen, alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate;
R₁₁ and R₁₂ are, independently for each occurrence, hydrogen, deuterium, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate; or R₁₁ and R₁₂ are, independently for each occurrence, selected from the group consisting of halogen, alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate; NR₁₃R₁₄ is optionally cyclized through C₃-C₁₂ cycloalkyl, or heterocyclyl, any of which are optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate;
If NR₁₃R₁₄ is not cyclized R₁₃ and R₁₄ are, independently for each occurrence, hydrogen, deuterium, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate, or R₁₃ and R₁₄ are, independently for each occurrence, selected from the group consisting of alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate;
Y is cyano, —C(O)R₁₅, —C(OH)R₁₅, —C(OR₁₆)R₁₅. R₁₅ and R₁₆ are, independently for each occurrence, hydrogen, deuterium, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate; or R₁₅ and R₁₆ are, independently for each occurrence, selected from the group consisting of alkyl ester, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate; and
* is a stereocenter selected from (R)-, (S)- or (R,S)-configuration, wherein the Stereocenter is in an R configuration, and wherein the compound is present in greater than 90%, 95%, 97%, 99%, 99.5%, or 99.9% enantiomeric excess (e.e) compared to the S isomer, or wherein the stereocenter is in an S configuration, and wherein the compound is present in greater than 90%, 95%, 97%, 99%, 99.5%, or 99.9% enantiomeric excess (e.e) compared to the R isomer.

### EXAMPLES

The compound structures in the examples below were confirmed by one or more of the following methods: ¹H NMR, ¹³C NMR, mass spectrometry (LC-MS), FTIR spectroscopy, HPLC-UV, Chiral-HPLC. ¹H NMR spectra were determined using an NMR spectrometer operating at 300 MHz or 400 MHz field. Chemical shifts were referenced to signals from residual protons in deuterated solvents as follows: CDCl₃=7.25 ppm, DMSO-d₆=2.49 ppm, CD₃OD=3.30 ppm. Peak multiplicities are designated as follows: s, singlet; d, doublet; dd, doublet of doublets; t, triplet; dt, doublet of triplets; q, quartet; br, broadened; and m, multiplet. Coupling constants are given in Hertz (Hz).

Mass spectra (MS) data are obtained using a mass spectrometer with ESI ionization and Ion trap or TOF mass analyzer.

In the examples below, reagents and solvents may be purchased from commercial suppliers, and may be used without further purification unless otherwise indicated.

### Example 1: benzyl (S)-4-methyl-L2,3-oxathiazolidine-3-carboxylate 2,2-dioxide ((S)-1′)

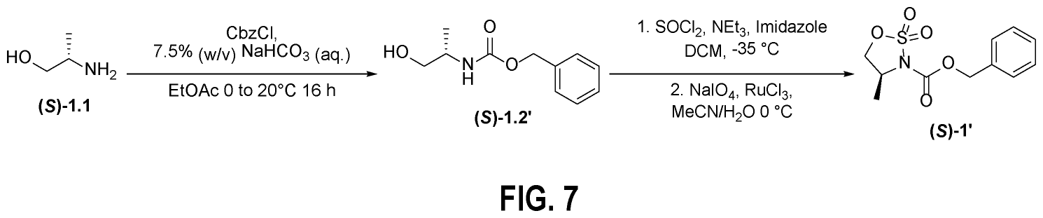

This Example 1 describes a two-step method that was used to prepare benzyl (S)-4-methyl-1,2,3-oxathiazolidine-3-carboxylate 2,2-dioxide ((S)-1′), as shown in FIG. 7.

### Step 1: Cbz Protection of (S)-2-aminopropan-1-ol ((S)-1.1) to Form benzyl (S)-(1-hydroxypropan-2-yl)carbamate ((S)-1.2′)

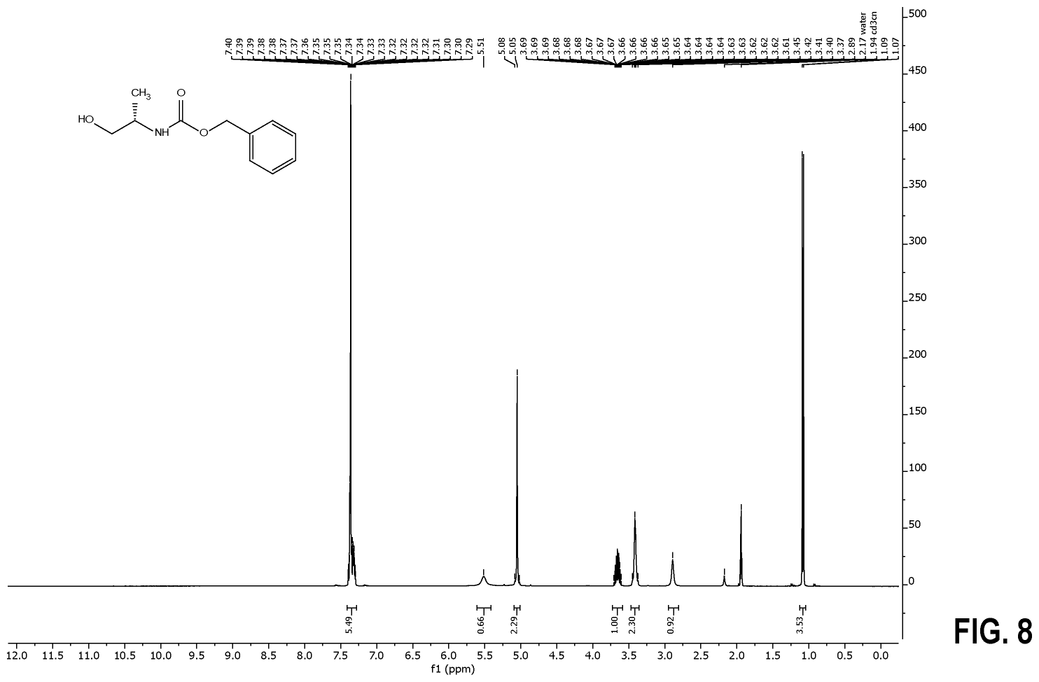

To a 5 L round bottom flask equipped with an overhead mechanical stirrer, thermocouple, N₂ inlet, and addition funnel was added (S)-2-aminopropan-1-ol ((S)-1.1) (100.0 g, 1331 mmol, 1.0 equiv.), EtOAc (1000 mL, 10 vol.), and 7.5% (w/v) NaHCO₃ (aq.) (1000 mL, 10 vol.). The stirring solution was cooled to 0° C. via an ice-water bath and CbzCl (209.0 mL, 1460 mmol, 1.1 equiv.) was added over 25 min via addition funnel. The biphasic reaction was allowed to equilibrate to an internal temperature of 20° C. over 16 h. Upon reaction completion, as determined by consumption of (S)-1.1 by ¹H-NMR and TLC (DCM:MeOH [9:1 ratio] using ceric ammonium molybdate stain, Rf=˜0.6), stirring was discontinued and the biphasic mixture was allowed to settle. The organic fraction was separated, and the aqueous fraction was extracted further with IPAc (2×500 mL, 5 vol.). The combined organic layers were washed with 36% (w/v) NaCl (aq) (100 mL, 1 vol.) and evaporated under reduced pressure to provide a clear colorless oil. The crude material was diluted with IPAc (2.8 V, 280-mL) and warmed to 45° C. with rotary agitation. Dissolution was not complete after 45 minutes, so additional IPAc (2.8 V, 280-mL) was added and heating with stirring was continued. Dissolution was complete after 1 h. Heating was discontinued, and the solution was seeded with authentic ((S)-1.2′) (approx. 100 mg). Moderate stirring (200-250 rpm) was continued while equilibrating to 20° C. over 16 h to provide a free-flowing slurry. The solids were collected via vacuum filtration and dried under reduced pressure at 45° C. to provide benzyl (S)-(1-hydroxypropan-2-yl)carbamate ((S)-1.2′) as a white solid (185.9 g, 67% yield) from a single crop. An additional crystallization attempt was performed via concentration of the mother liquor followed by seeding with authentic ((S)-1.2′) to provide a free-flowing slurry that was subsequently filtered and dried under reduced pressure to give an additional 21.4 g of benzyl (S)-(1-hydroxypropan-2-yl)carbamate ((S)-1.2′) as a white solid. Combined total amount isolated after the second crop: 207.3 g, 74% yield. ¹H NMR (400 MHz, Acetonitrile-d₃) δ 7.41-7.28 (m, 5H), 5.51 (s, 1H), 5.10-5.01 (m, 2H), 3.72-3.59 (m, 1H), 3.47-3.35 (m, 2H), 2.89 (s, 1H), 1.08 (d, J=6.8 Hz, 3H), as shown in FIG. 8.

### Step 2: Two-Step/One-Pot Cyclization/Oxidation to Form benzyl (S)-4-methyl-1,2,3-oxathiazolidine-3-carboxylate 2,2-dioxide ((S)-1′)

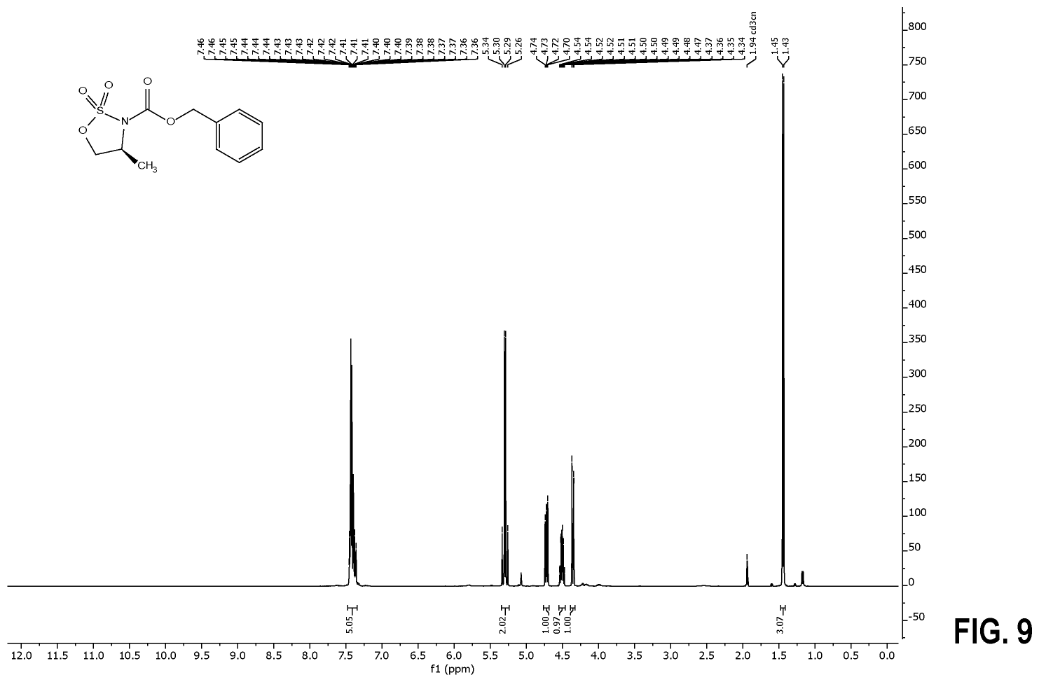

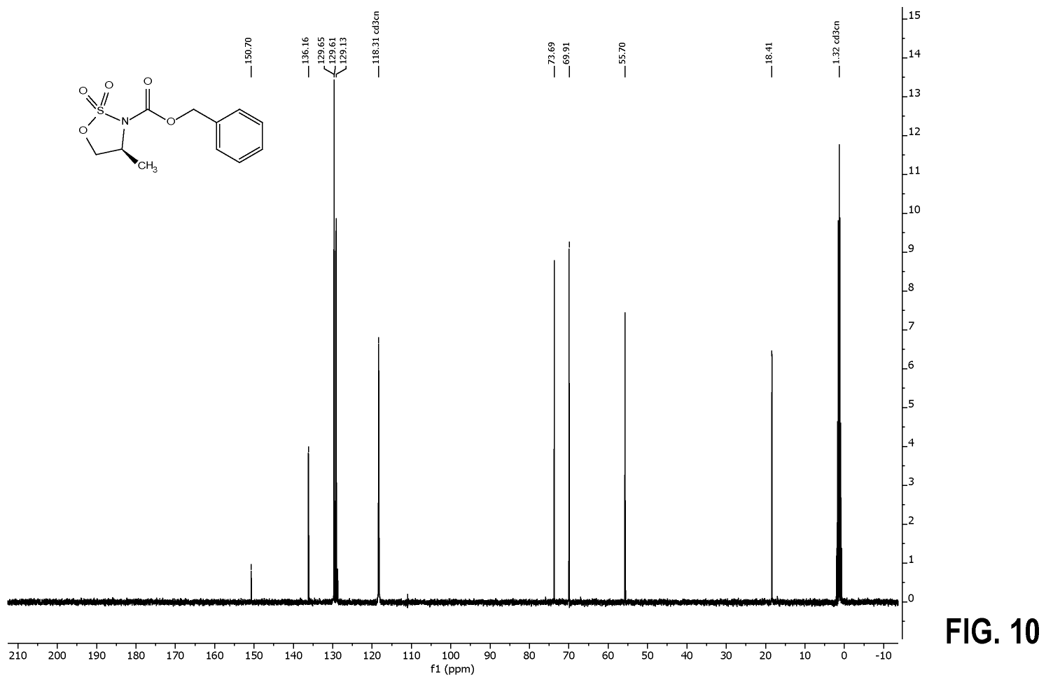

To a 400 mL Easy Max Reactor equipped with an overhead mechanical stirrer, thermocouple, and N₂ inlet was added imidazole (32.54 g, 477.9 mmol, 4.0 equiv.), DCM (100 mL, 4 vol.), and NEt₃ (37 mL, 262.8 mmol, 2.2 equiv.). The vessel was purged with N₂ for 30 min and cooled to an internal temperature of −35° C. To the stirring suspension was added a solution of thionyl chloride (9.5 mL, 131.4 mmol, 1.1 equiv.) in DCM (50 mL, 2 vol.) over 2 h, via syringe pump, and allowed to stir for an additional 15 min. To the resultant suspension was added a solution of benzyl (S)-(1-hydroxypropan-2-yl)carbamate ((S)-1.2′) (25.00 g, 119.5 mmol, 1.0 equiv.) in DCM (100 mL, 4 vol.) over 1 h, via syringe pump. The reaction was warmed to −30° C. and allowed to stir for 15 h. Upon reaction completion, as determined by consumption of 2 by HPLC and ¹H NMR, the reaction was warmed to 0° C. over 2 h. The reaction was washed with H₂O (2×10 vol.) and the organic fraction was concentrated under reduced pressure. The crude oil was reconstituted in MeCN (100 mL, 4 vol.) and H₂O (125 mL, 5 vol.) then cooled to an internal temperature of 0° C. To the stirring solution was added NaIO₄ (28.11 g, 131.4 mmol, 1.1 equiv.) followed by RuCl₃ (25 mg, 0.12 mmol, 0.001 equiv.) and the solution was agitated for 3 h at 0° C. Upon reaction completion, as determined by consumption of the intermediate sulfamidite by HPLC and ¹H NMR, the reaction was treated with a pH=7 KH₂PO₄/KOH buffer (aq) (250 mL, 10 vol.) and PhMe (100 mL, 4 vol.). The MeCN was removed under reduced pressure and the organic and aqueous fractions were separated. The aqueous fraction (pH=7) was extracted further with IPAc (2×4 vol.), and the combined organic layers were concentrated under reduced pressure to an estimated 50 mL (˜2 V). The solution was warmed to 45° C. and seeded with authentic (S)-3-Cbz-4-methyl-1,2,3-oxathiazolidine 2,2-dioxide 3. To the stirring suspension was added n-heptane (250 mL, 10 vol.) dropwise over 1 h to provide a white slurry. The suspension was cooled and allowed to stir at 20° C. for 1 h, gradually cooled to −20° C. over 2 h, then stirred for an additional 15 h. The slurry was filtered, and the solids were washed with n-heptane (4 vol.). The collected solids were dried under reduced pressure at 20° C. to provide benzyl (S)-3-Cbz-4-methyl-1,2,3-oxathiazolidine 2,2-dioxide ((S)-1′) as a white solid (27.17 g, 84% yield). ¹H NMR (400 MHz, Acetonitrile-d₃) δ 7.48-7.34 (m, 5H), 5.36-5.23 (m, 2H), 4.72 (dd, J=9.4, 5.8 Hz, 1H), 4.56-4.45 (m, 1H), 4.35 (dd, J=9.4, 2.3 Hz, 1H), 1.44 (d, J=6.4 Hz, 3H), as shown in FIG. 9. ¹³C NMR (101 MHz, Acetonitrile-d₃) δ 150.70, 136.16, 129.65, 129.61, 129.13, 73.69, 69.91, 55.70, 18.41, as shown in FIG. 10.

### Example 2: tert-butyl (R)-2-methylaziridine-1-carboxylate ((R)-2″)

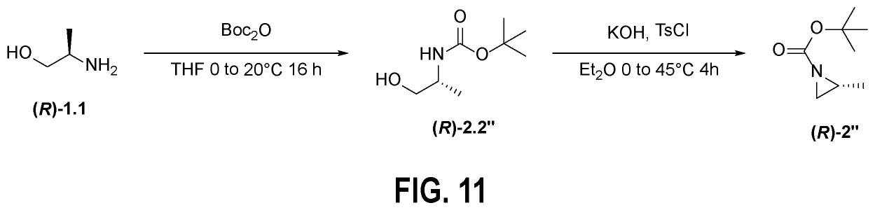

This Example 2 describes a two-step method that was used to prepare tert-butyl (R)-2-methylaziridine-1-carboxylate ((R)-2″), as shown in FIG. 11.

Step 1: Boc protection of (R)-2-aminopropan-1-ol ((R)-1.1) to form tert-butyl (R)-(1-hydroxypropan-2-yl)carbamate ((R)-2.2″). This step was carried out by following literature procedures (see WO 2014/159224, incorporated by reference herein in its entirety) with modifications.

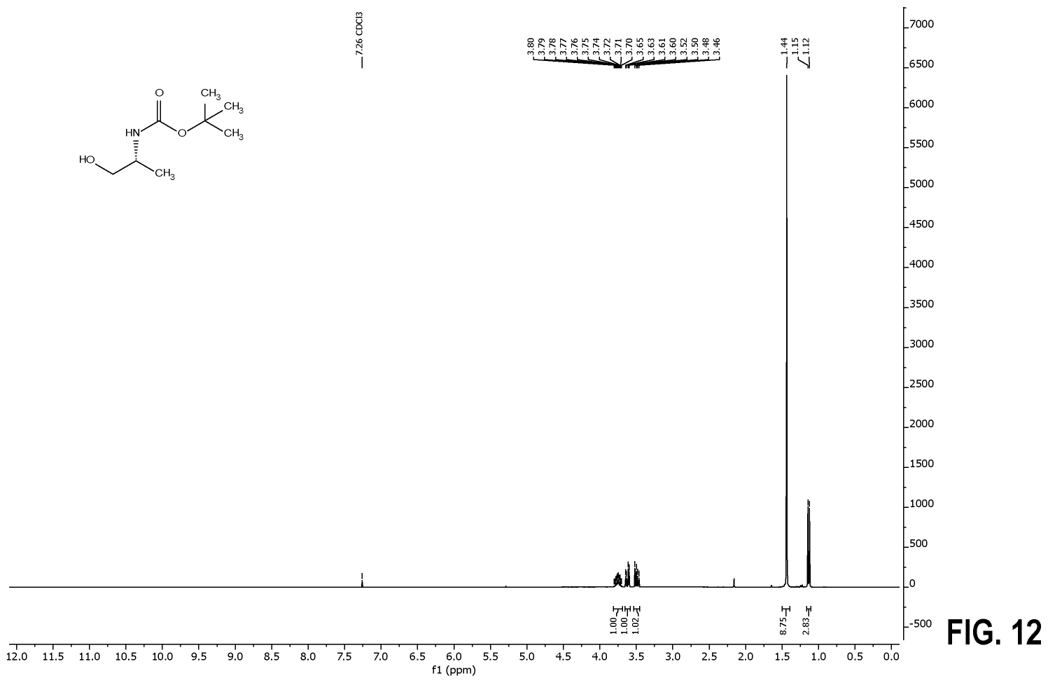

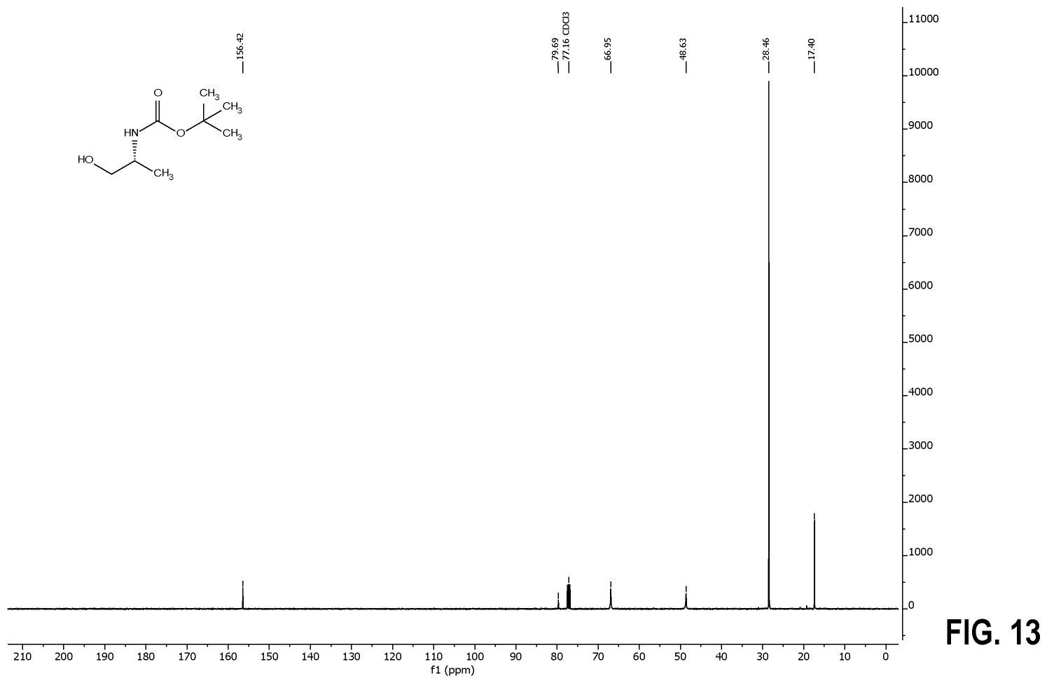

To a 250 mL round bottom flask under N₂ atmosphere was added (R)-2-aminopropan-1-ol ((R)-1.1) (10.0 g, 133.1 mmol, 1.0 equiv.) and anhydrous THF (100 mL). The flask was cooled to 0° C. and a solution of Boc₂O (30.52 g, 139.8 mmol, 1.05 eq.) in anhydrous THF (40 mL) was added dropwise over 30 minutes. The reaction mixture was stirred at 0° C. for a further 1 h and then warmed to 20° C. over a further 16 h. Upon reaction completion, as determined by consumption of (R)-1.1 by ¹H-NMR and TLC (DCM:MeOH [9:1 ratio] using potassium permanganate stain, Rf=˜0.6), stirring was discontinued. The organics were concentrated under reduced pressure and the residue was triturated with n-hexane. The resulting solid was filtered and air dried to give the title compound ((R)-2.2″) as a colorless solid (20.81 g, 89% yield). HRMS (ESI): m/z calculated for C₈H₁₇NO₃+Na⁺ [M+Na]⁺: 198.1101. Found: 198.0791. ¹H NMR (300 MHz, CDCl₃) δ 3.81-3.69 (m, 1H), 3.62 (dd, J=10.9, 3.9 Hz, 1H), 3.49 (dd, J=10.9, 6.1 Hz, 1H), 1.44 (s, 9H), 1.14 (d, J=6.8 Hz, 3H) ppm as shown in FIG. 12. ¹³C NMR (101 MHz, CDCl₃) δ 156.42, 79.69, 66.95, 48.63, 28.46, 17.40 ppm as shown in FIG. 13.

Step 2: Two-step/one-pot tosylation/cyclization to form tert-butyl (R)-2-methylaziridine-1-carboxylate ((R)-2″). This step was carried out by following literature procedures (see WO 2012/14109, incorporated by reference herein in its entirety) with modifications.

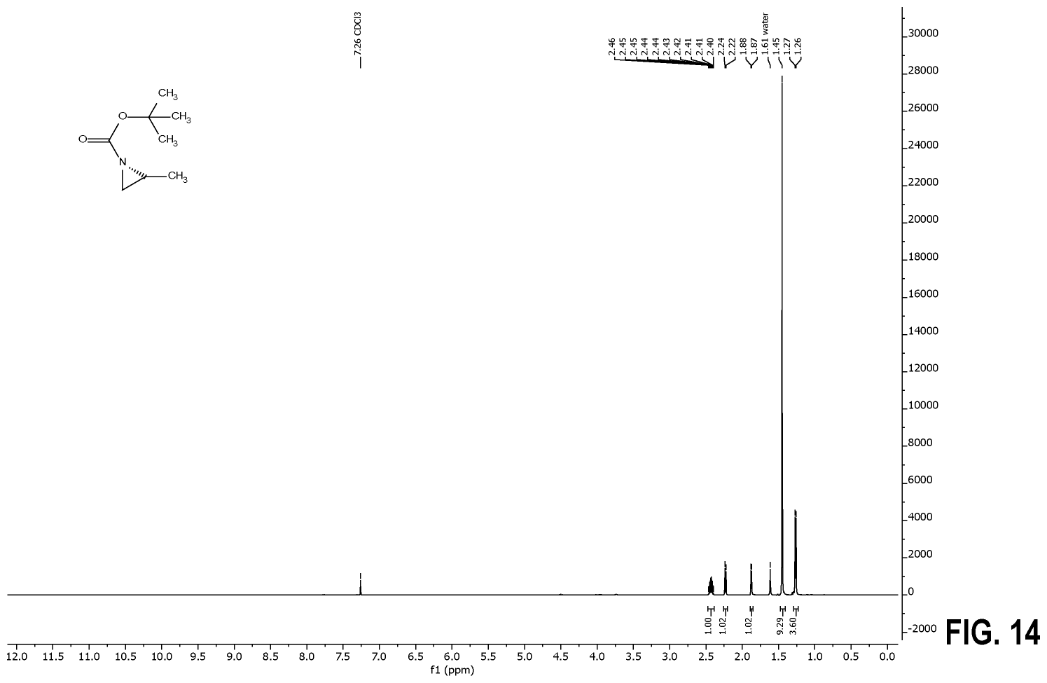

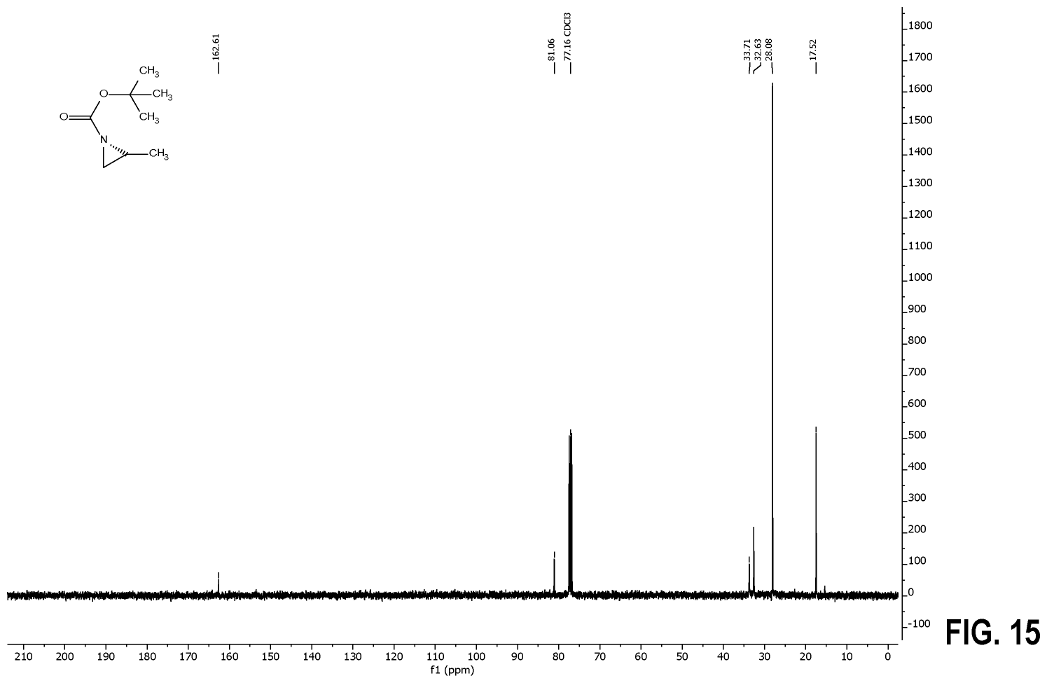

To a 250 mL round bottom flask under N₂ atmosphere was added tert-butyl (R)-(1-hydroxypropan-2-yl)carbamate ((R)-2.2″) (10.00 g, 57.1 mmol, 1 equiv.), anhydrous diethyl ether (120 ml) and tosyl chloride (14.14 g, 74.2 mmol, 1.3 equiv.). The reaction mixture was stirred for 15 minutes and then cooled to 0° C. Crushed potassium hydroxide (25.62 g, 456.8 mmol, 8 equiv.) was added portion-wise over 30 minutes. The reaction mixture was warmed at 45° C. for about 3 hours, and upon reaction completion, as determined by consumption of (R)-2.2′ by ¹H-NMR and TLC (Hex:EtOAc [8:2 ratio] using potassium permanganate stain, Rf=˜0.3), stirring was discontinued. Reaction mixture was diluted with water (100 mL), the organic fraction was separated, and the aqueous fraction was extracted further with diethyl ether (2×50 mL). The combined organic layers were washed with 36% (w/v) NaCl (aq) (100 mL) and evaporated under reduced pressure to provide tert-butyl (R)-2-methylaziridine-1-carboxylate ((R)-2″) as a clear colorless oil (7.04 g, 78% yield). ¹H NMR (400 MHz, CDCl₃) δ 2.48-2.39 (m, 1H), 2.23 (d, J=5.8 Hz, 1H), 1.88 (d, J=3.8 Hz, 1H), 1.45 (s, 9H), 1.27 (d, J=5.5 Hz, 3H) ppm as shown in FIG. 14. ¹³C NMR (101 MHz, CDCl₃) δ 162.61, 81.06, 33.71, 32.63, 28.08, 17.52 ppm as shown in FIG. 15.

### Example 3: (S)-4-(dimethylamino)-2,2-diphenylpentanenitrile ((S)-5)

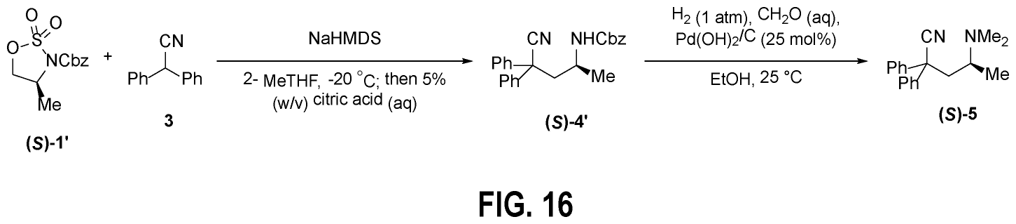

This Example 3 describes a two-step method that was used to prepare (S)-4-(dimethylamino)-2,2-diphenylpentanenitrile ((S)-5), as shown in FIG. 16.

### Step 1: Ring-opening reaction of benzyl (S)-4-methyl-1,2,3-oxathiazolidine-3-carboxylate 2,2-dioxide ((S)-1′) on the 2,2-diphenylacetonitrile (3) anion to form benzyl (S)-(4-cyano-4,4-diphenylbutan-2-yl)carbamate ((S)-4′)

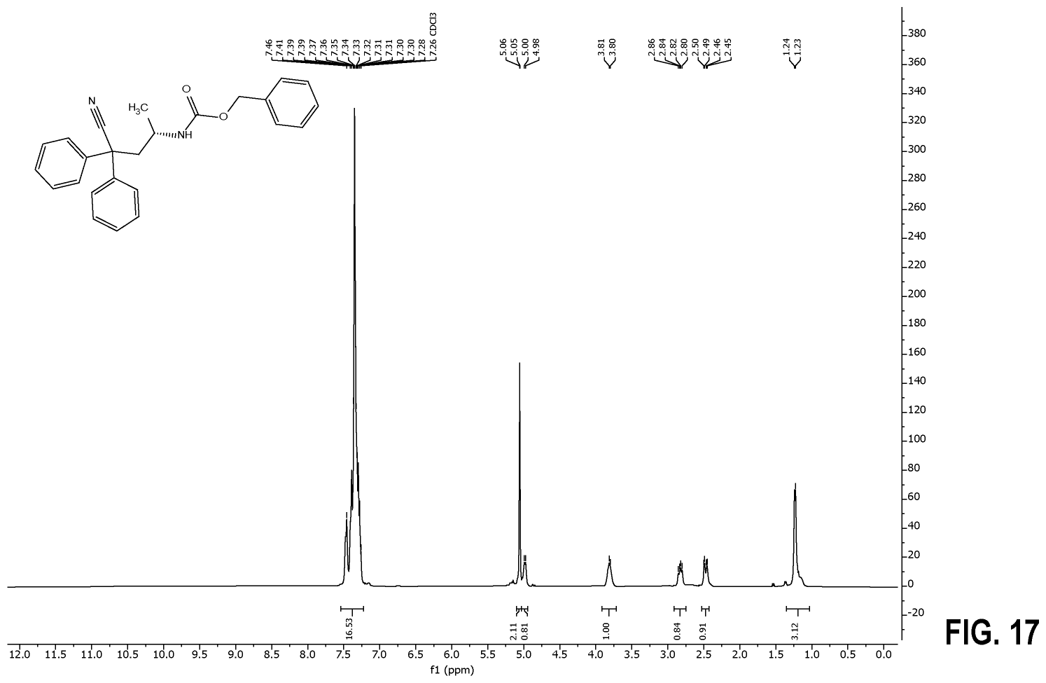

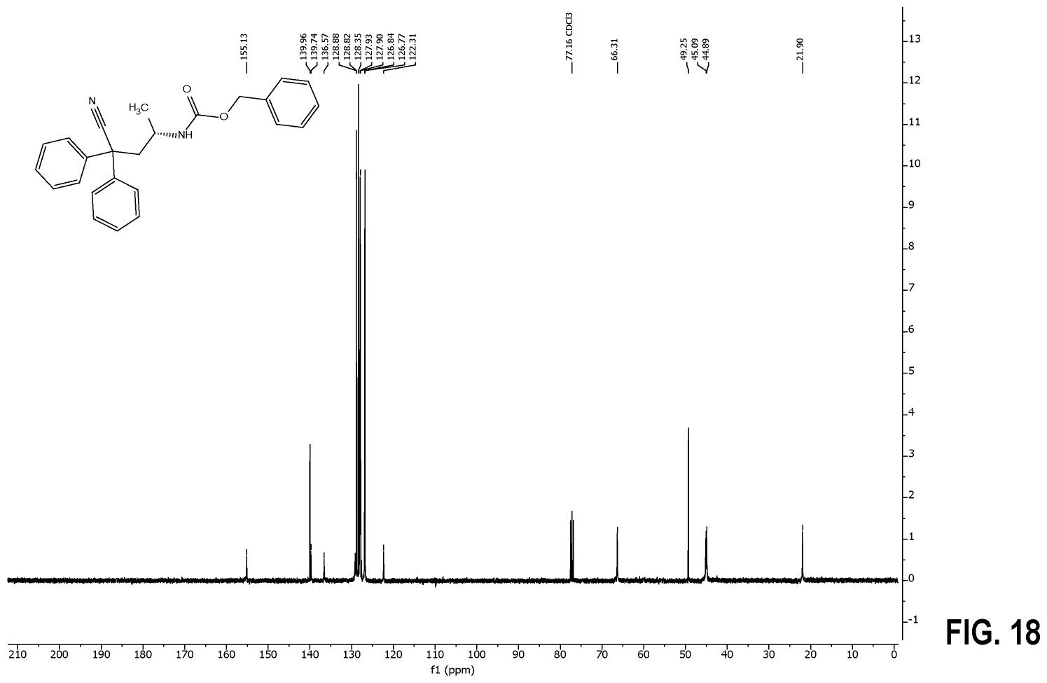

To a 400 mL Easy Max Reactor equipped with an overhead mechanical stirrer, thermocouple, and N₂ inlet was added benzyl (S)-4-methyl-1,2,3-oxathiazolidine-3-carboxylate 2,2-dioxide ((S)-1′) (10.00 g, 36.9 mmol, 1 equiv.) and diphenylacetonitrile (3) (7.85 g, 40.6 mmol, 1.1 equiv.). The vessel was purged with N₂ for 30 min, diluted with 2-MeTHF (100 mL, 10 vol.), and cooled to an internal temperature of −20° C. To the stirring solution was added NaHMDS (1 M in THF, 44.3 mL, 44.3 mmol, 1.2 equiv.) dropwise over 1 h, via syringe pump, and allowed to stir for an additional 15 h. The mixture was warmed to 0° C. and stirred for 1 h. Upon reaction completion, as determined by consumption of (S)-1′ by HPLC, the solution was treated with 5% (w/v) citric acid (aq.) (50 mL, 5 vol.) added dropwise over 1 h. The biphasic mixture was warmed to 20° C. then the organic and aqueous fractions were separated. The aqueous fraction was extracted further with 2-MeTHF (2×50 mL, 5 vol.). The combined organic layers were concentrated under reduced pressure to an estimated 20 mL (˜2 vol.) at 35° C. To the stirring solution was added n-heptane (100 mL, 10 vol.), dropwise over 1 h, then seeded with authentic benzyl (S)-(4-cyano-4,4-diphenylbutan-2-yl)carbamate ((S)-4′) to provide an off-white slurry. The slurry was allowed to stir at 20° C. for 6 h, gradually cooled to −20° C. over 15 h (˜3° C./h), then stirred for an additional 15 h. The slurry was filtered, and the solids were washed with n-heptane (3×10 vol.). The collected solids were dried under reduced pressure at 20° C. to provide benzyl (S)-(4-cyano-4,4-diphenylbutan-2-yl)carbamate ((S)-4′) as a white solid (13.62 g, 96% yield). Purity HPLC: 97.9%. HRMS (ESI) m/z: [M+H]⁺ Required C₂₅H₂₄N₂O₂ 385.19; Found 385.20. ¹H NMR (400 MHz, CDCl₃) δ 7.51-7.20 (m, 15H), 5.06 (s, 2H), 4.99 (d, J=8.3 Hz, 1H), 3.86-3.74 (m, 1H), 2.83 (dd, J=14.4, 8.0 Hz, 1H), 2.48 (dd, J=14.4, 4.7 Hz, 1H), 1.24 (d, J=6.7 Hz, 3H) ppm, as shown in FIG. 17. ¹³C NMR (400 MHz, CDCl₃) δ 155.13, 139.96, 139.74, 136.57, 128.88, 128.82, 128.35, 127.93, 127.90, 126.84, 126.77, 122.31, 66.31, 49.25, 45.09, 44.89, 21.90 ppm, as shown in FIG. 18.

### Step 2: Two-Step/One-Pot Deprotection/Reductive Amination of benzyl (S)-(4-cyano-4,4-diphenylbutan-2-yl)carbamate ((S)-4′) to form (S)-methadone nitrile ((S)-5)

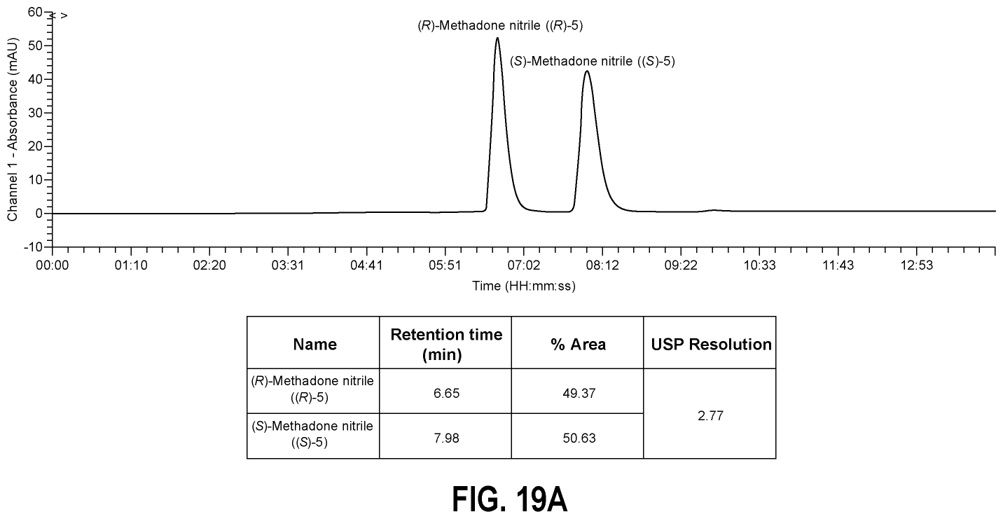

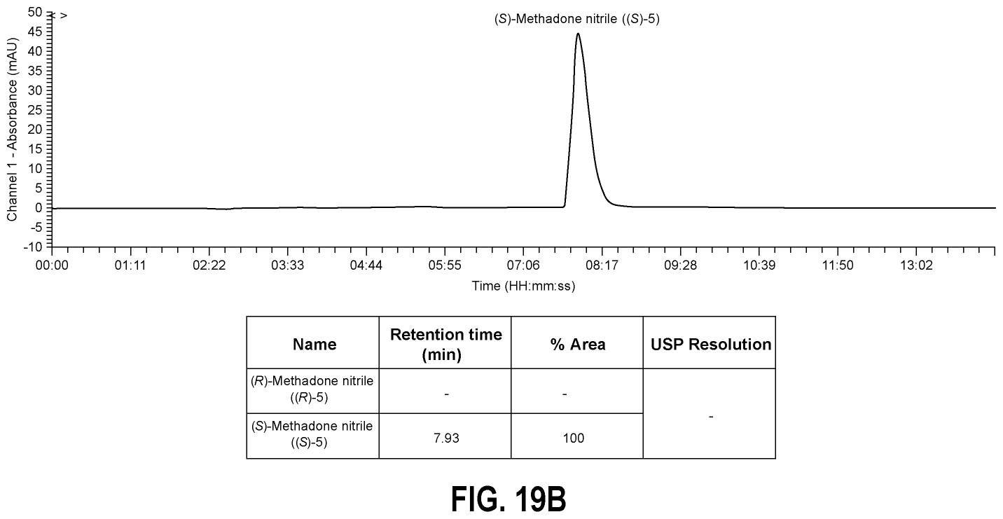

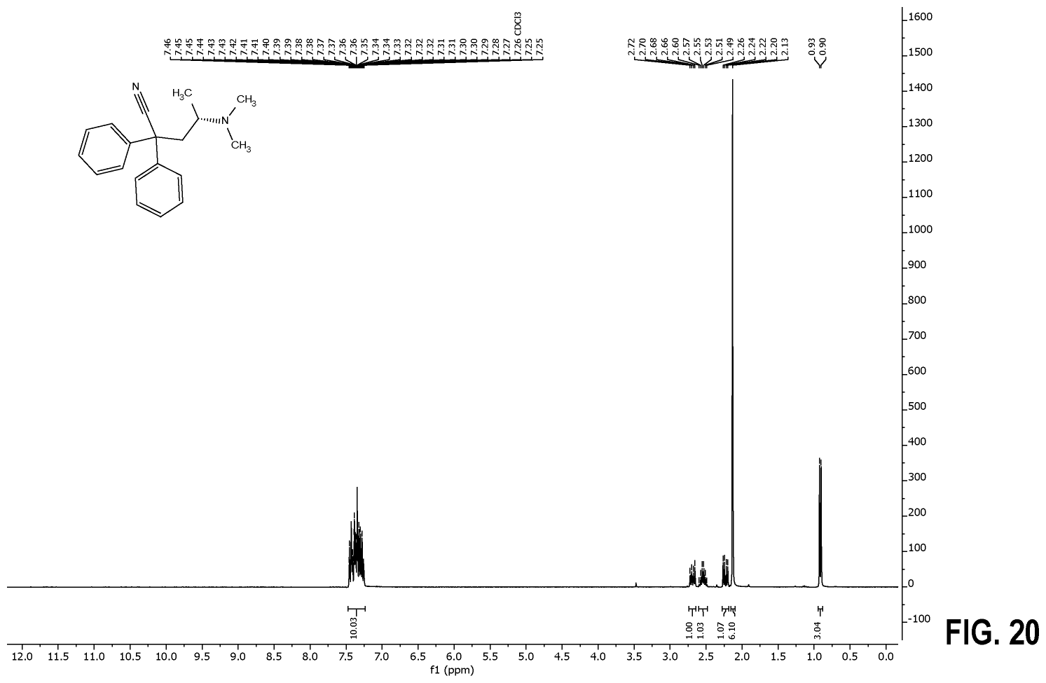

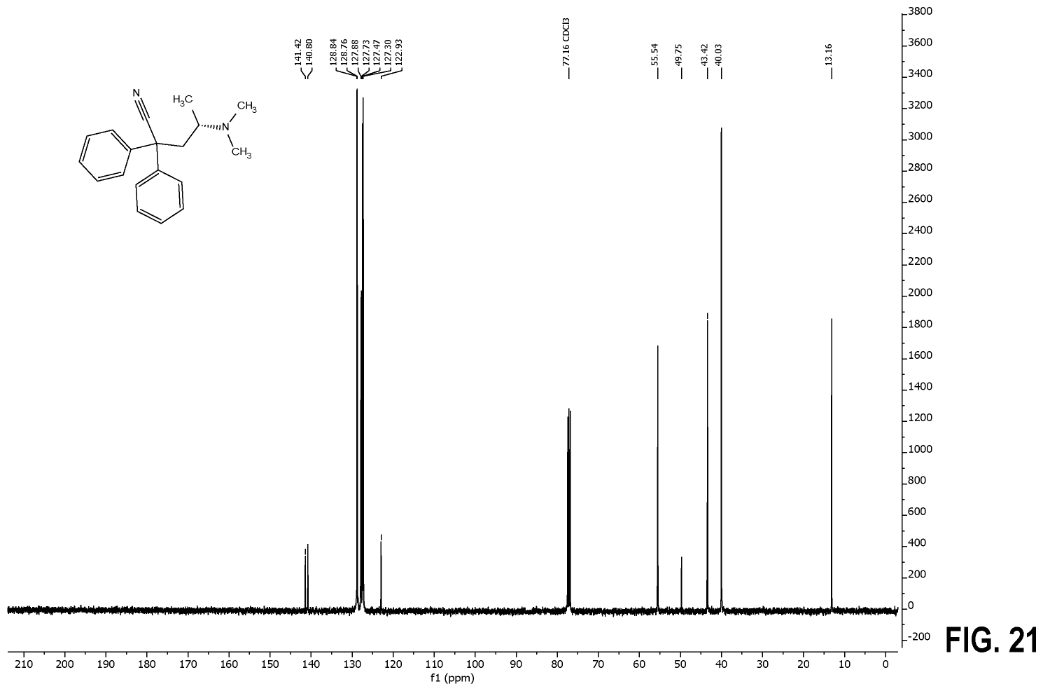

To a 100 mL Morton Type round bottom flask equipped with a magnetic stir bar was added benzyl (S)-(4-cyano-4,4-diphenylbutan-2-yl)carbamate ((S)-4′) (1.00 g, 2.60 mmol, 1 equiv.) and Pd(OH)₂/C 20% on activated charcoal (460 mg, 0.65 mmol, 25 mol %). The vessel was purged with N₂ for ˜30 min, diluted with EtOH (20 mL, 20 vol.), then to the stirring solution was added aqueous CH₂O 37% w/w (2 mL, 2 vol.). The vessel was equipped with a balloon of H₂ and allowed to stir at 25° C. for 24 h. Upon reaction completion, as determined by consumption of ((S)-4′) by HPLC, the vessel was purged with N₂ for ˜30 min. The suspension was filtered through a bed a celite eluting with EtOH (2×5 vol.) and concentrated under reduced pressure to ˜2 volumes. To the stirring ethanolic solution was added H2O (14 mL, 14 vol.) over 1 h and the slurry was allowed to stir at 25° C. for 15 h, cooled to −5° C. via an ice-water bath, then stirred for an additional 5 h. The solids were collected via vacuum filtration and washed with H₂O (5 mL, 5 vol.). The collected solids were dried under reduced pressure at 50° C. to provide (S)-4-(dimethylamino)-2,2-diphenylpentanenitrile ((S)-5) as a white solid (630 mg, 87% yield). Chiral HPLC: one single enantiomer (e.e. >99%) as shown in FIG. 19. HRMS (ESI) m/z: [M+H]⁺ Required C₁₉H₂₂N₂ 279.18; Found 279.20. ¹H NMR (300 MHz, CDCl₃) δ 7.49-7.22 (m, 10H), 2.69 (dd, J=13.7, 6.4 Hz, 1H), 2.54 (h, J=6.4 Hz, 1H), 2.23 (dd, J=13.6, 6.0 Hz, 1H), 2.13 (s, 6H), 0.91 (d, J=6.6 Hz, 3H). ppm, as shown in FIG. 20. ¹³C NMR (101 MHz, CDCl₃) δ 141.42, 140.80, 128.84, 128.76, 127.88, 127.73, 127.47, 127.30, 122.93, 55.54, 49.75, 43.42, 40.03, 13.16 ppm, as shown in FIG. 21.

### Example 4: (R)-4-(dimethylamino)-2,2-diphenylpentanenitrile ((R)-5)

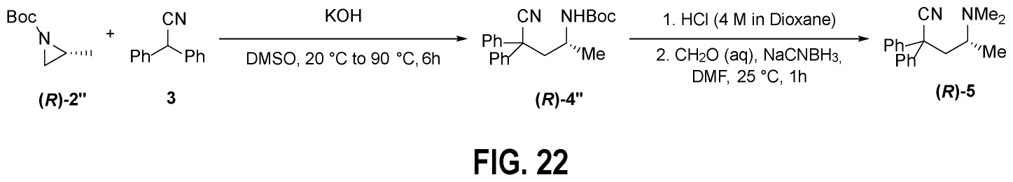

This Example 4 describes a two-step method that was used to prepare (R)-4-(dimethylamino)-2,2-diphenylpentanenitrile ((R)-5), as shown in FIG. 22.

### Step 1: Ring-Opening Reaction of tert-butyl (R)-2-methylaziridine-1-carboxylate ((R)-2″) on the 2,2-diphenylacetonitrile (3) anion to Form tert-butyl (R)-(4-cyano-4,4-diphenylbutan-2-yl)carbamate ((R)-4″)

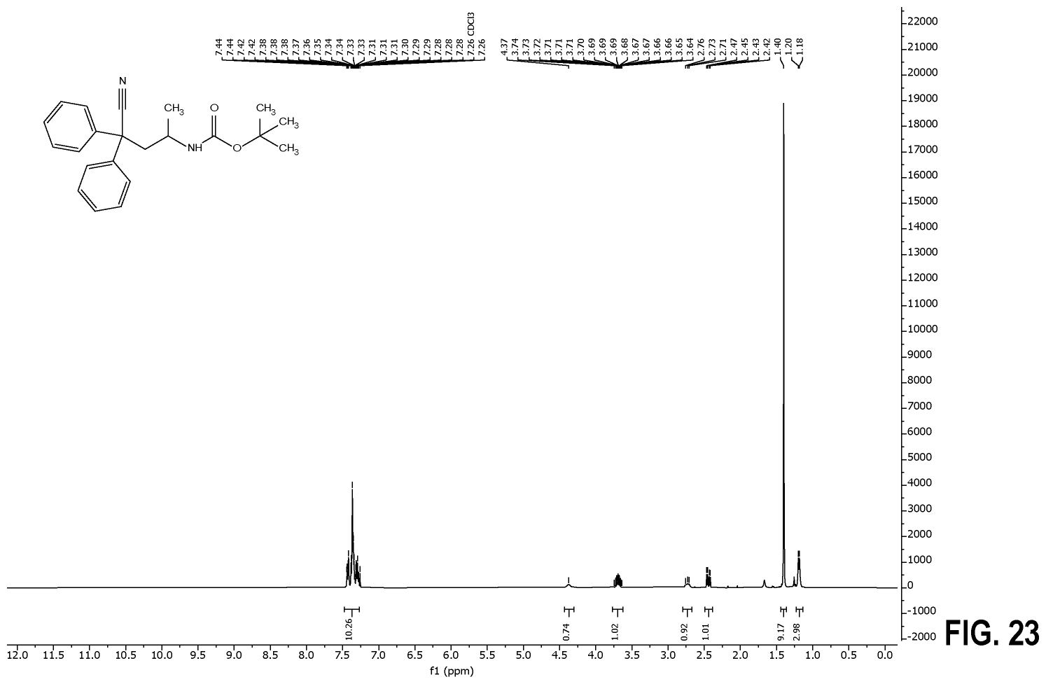

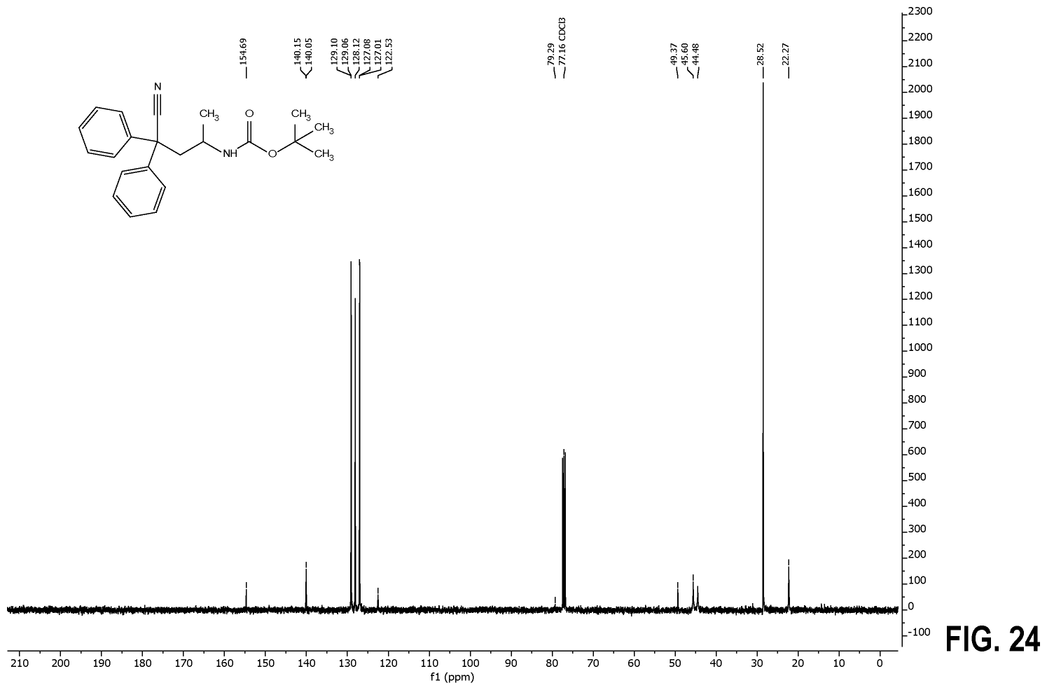

To a solution of KOH (35 mg) in anhydrous DMSO (1.44 mL) was added diphenylacetonitrile (3) (0.669 g, 3.5 mmol, 1.25 eq.) and a solution of tert-butyl (R)-2-methylaziridine-1-carboxylate ((R)-2″) (0.4365 g, 2.8 mmol, 1.0 eq.) in anhydrous DMSO (2.91 mL). The reaction mixture was stirred at 90° C. for 6 h. Upon reaction completion, as determined by consumption of (R)-2″ by ¹H-NMR and TLC (Hex:EtOAc [8:2 ratio] using potassium permanganate stain, Rf=˜0.6), stirring was discontinued. The resulting mixture was then poured into 50 mL of water/brine 1:1 and DCM (50 mL) was added. The organic layer was separated and the aqueous phase was further extracted with DCM (20 mL×3). The combined organic layers were evaporated under reduced pressure and the crude was purified by column chromatography using DCM/ethyl acetate 98:2 as eluent to obtain tert-butyl (R)-(4-cyano-4,4-diphenylbutan-2-yl)carbamate ((R)-4″) as a pale yellow solid (0.756 g, 78% yield). HRMS (ESI) m/z: [M+H]⁺ Required C₂₂H₂₆N₂O₂ 351.2067; Found: 351.2078. ¹H NMR (400 MHz, CDCl₃) δ 7.48-7.27 (m, 10H), 4.38 (s, 1H), 3.76-3.63 (m, 1H), 2.80-2.67 (m, 1H), 2.44 (dd, J=14.2, 5.2 Hz, 1H), 1.40 (s, 9H), 1.19 (d, J=6.6 Hz, 3H) ppm, as shown in FIG. 23. ¹³C NMR (101 MHz, CDCl₃) δ 154.69, 140.15, 140.05, 129.10, 129.06, 128.12, 127.08, 127.01, 122.53, 79.29, 49.37, 45.60, 44.48, 28.52, 22.27 ppm, as shown in FIG. 24.

### Step 2: Two-Step/One-Pot Deprotection/Reductive Amination of tert-butyl (R)-(4-cyano-4,4-diphenylbutan-2-yl)carbamate ((R)-4″) to form (R)-methadone nitrile ((R)-5)

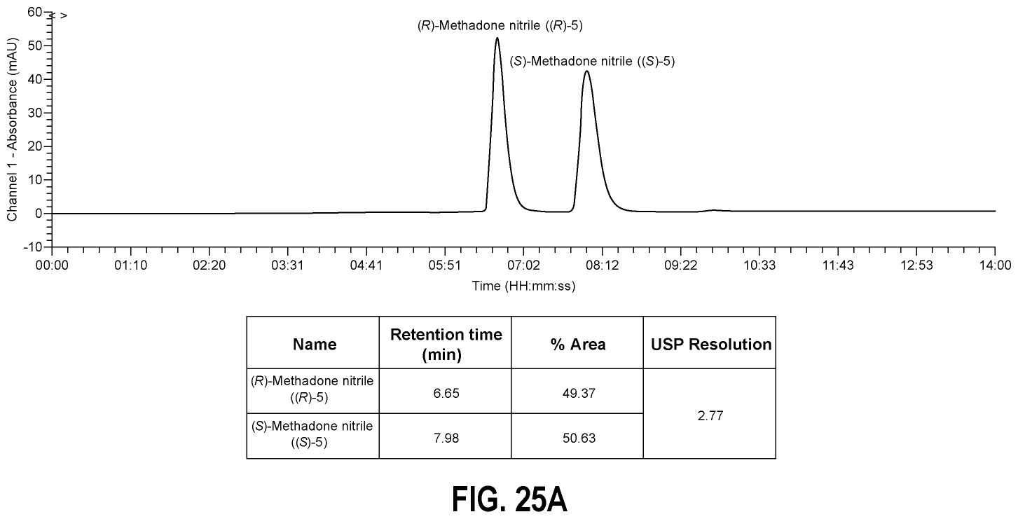

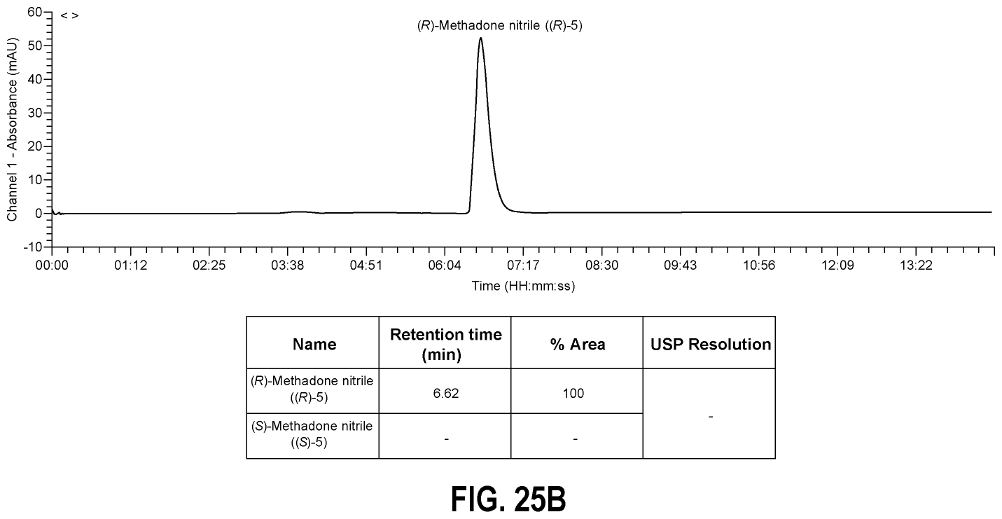

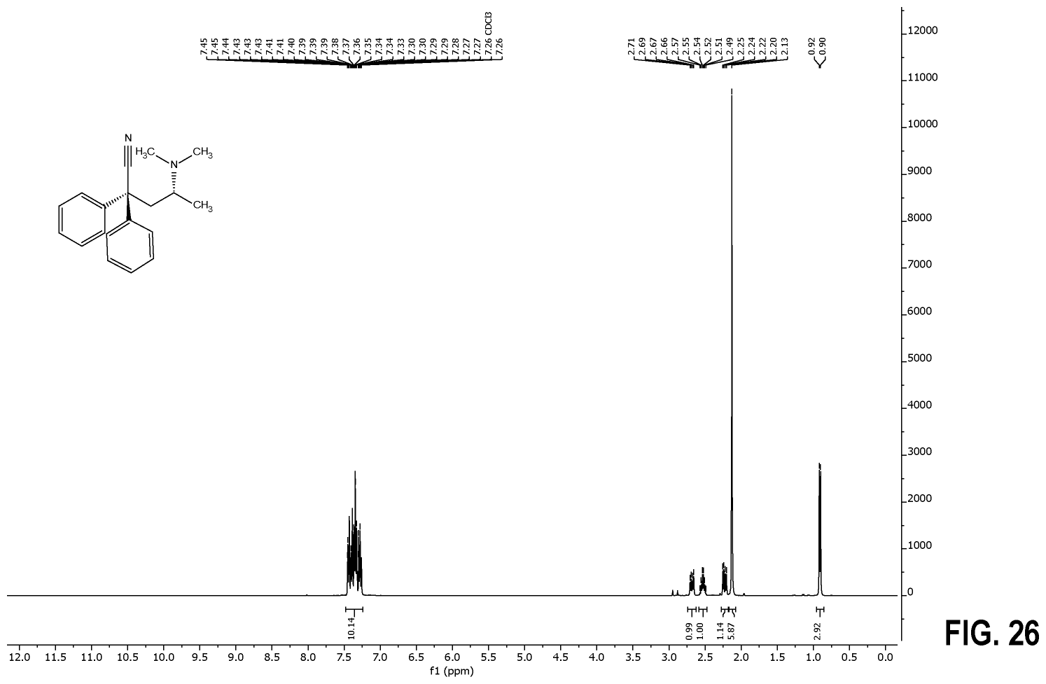

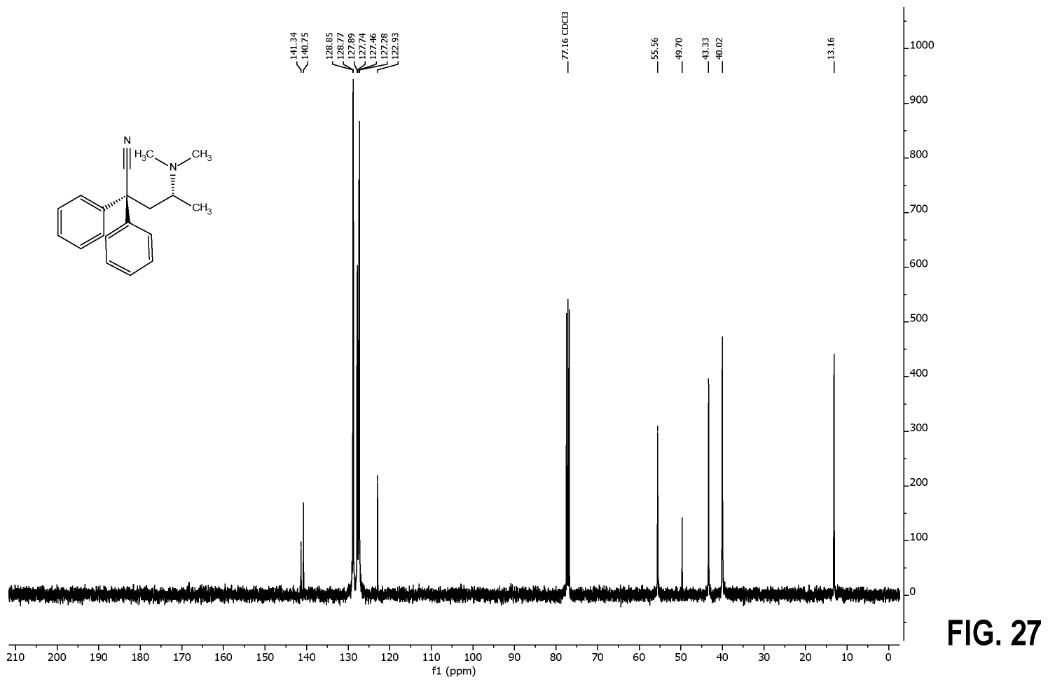

To an ice-cooled solution of tert-butyl (R)-(4-cyano-4,4-diphenylbutan-2-yl)carbamate ((R)-4″) (0.200 g, 0.57 mmol, 1 eq.) in DCM (2 mL) was added HCl (4M in dioxane, 2 mL, 14 mmol, 25 eq.) dropwise under nitrogen atmosphere and the mixture was stirred at 0° C. for 3 h. Then, the solvents and residual HCl were removed under reduced pressure to obtain a slightly yellow oil of Boc-deprotected (R)-4″ which was redissolved in DMF (4.3 mL) and cooled in ice-bath. Formaldehyde (37% in water, 1.15 mL, 15 mmol, 25 eq.) and NaCNBH₃ (76 mg, 1.22 mmol, 2 eq) were added and the mixture was stirred at rt for 1 h. The solution was then poured into 50 mL of sat. NaHCO₃ and extracted with DCM (3×50 mL). The combined organic fractions were evaporated under reduced pressure, and the crude was purified by column chromatography using CHCl₃/Methanol 95:5 as eluent to obtain (R)-4-(dimethylamino)-2,2-diphenylpentanenitrile ((R)-5) (138 mg, 87% yield). Chiral HPLC: one single enantiomer (e.e. >99%) as shown in FIG. 25. HRMS (ESI) m/z: [M+H]⁺ Required C₁₉H₂₂N₂ 279.1861; Found 279.1924. ¹H NMR (400 MHz, CDCl₃) δ 7.48-7.24 (m, 10H), 2.68 (dd, J=13.8, 6.5 Hz, 1H), 2.59-2.47 (m, 1H), 2.23 (dd, J=13.9, 6.0 Hz, 1H), 2.13 (s, 6H), 0.91 (d, J=6.6 Hz, 3H) ppm, as shown in FIG. 26. ¹³C NMR (101 MHz, CDCl₃) δ 141.34, 140.75, 128.85, 128.77, 127.89, 127.74, 127.46, 127.28, 122.93, 55.56, 49.70, 43.33, 40.02, 13.16 ppm, as shown in FIG. 27.

### Example 5: (S)-6-(Dimethylamino)-4,4-Diphenyl-3-Heptanone Hydrochloride ((S)-Methadone*HCl)

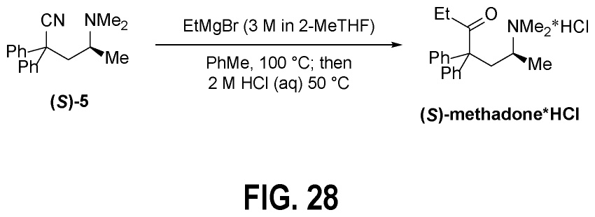

This Example 5 describes a one-step method that was used to prepare (S)-6-(dimethylamino)-4,4-diphenyl-3-heptanone hydrochloride ((S)-methadone*HCl), as shown in FIG. 28. This step was carried out by following literature procedures (see WO 2017/35224 and US2020/277250, incorporated by reference herein in their entireties) with modifications.

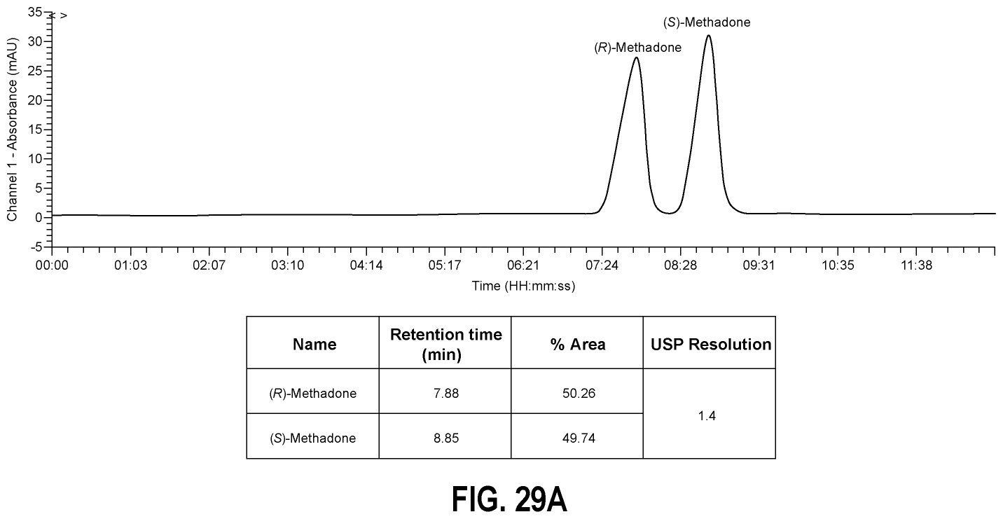

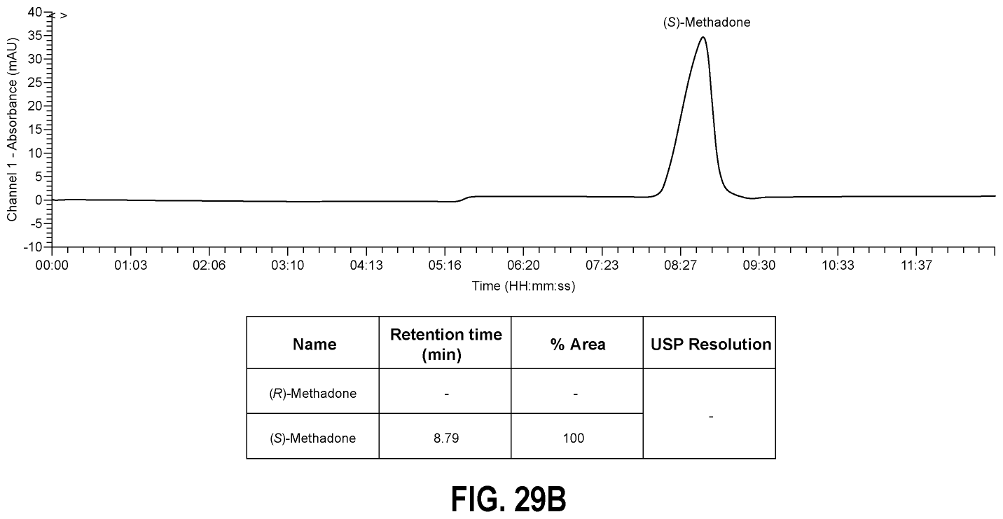

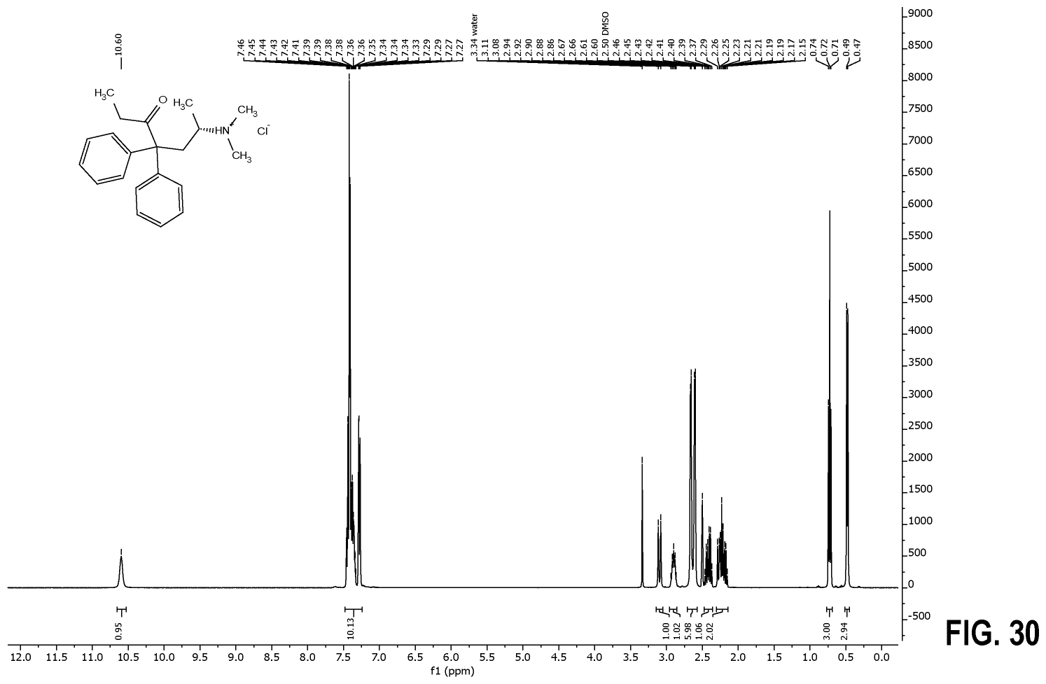

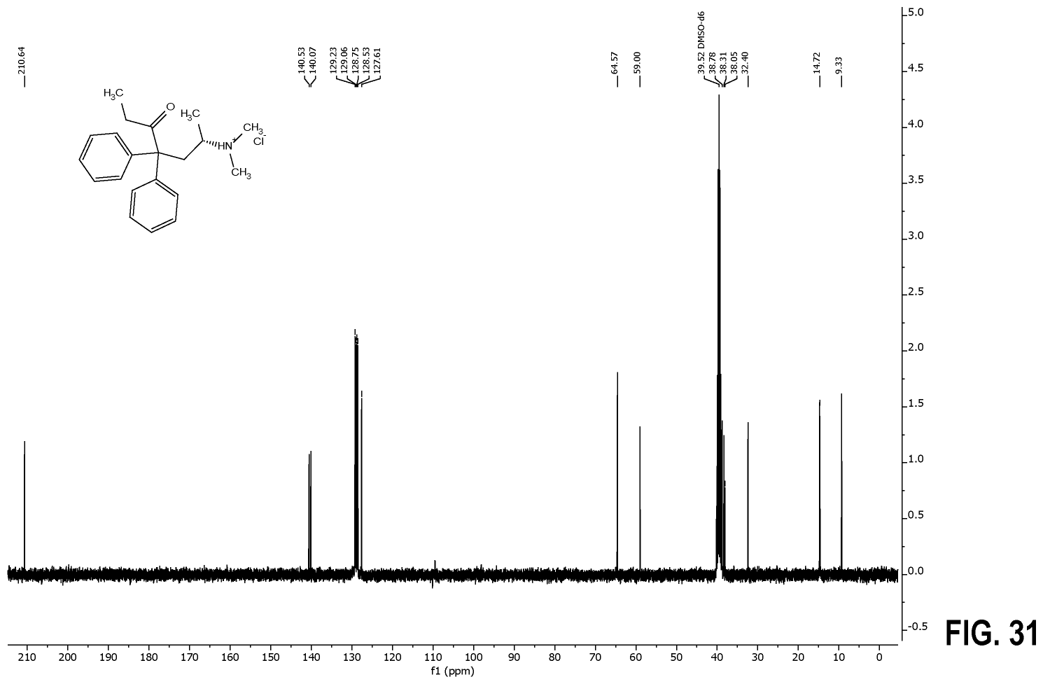

To a 200 mL two-neck round bottom flask equipped with a thermocouple, magnetic stir bar, and N₂ inlet was added (S)-4-(dimethylamino)-2,2-diphenylpentanenitrile ((S)-5) (6.62 g, 23.78 mmol, 1 equiv.). The vessel was purged with N₂ for 30 min, diluted with PhMe (66 mL, 10 vol.), and to the resultant solution was added 3 M EtMgBr in 2-MeTHF (12 mL, 35.68 mmol, 1.5 equiv.) dropwise over 30 min. The reaction mixture was then warmed to 100° C. via a heating mantle and allowed to stir for 24 h. Upon consumption of (S)-5 as determined by the HPLC, the solution was cooled to 0° C., and charged with 2 M aqueous HCl (13 mL, 2 vol.). The biphasic mixture was then warmed to 50° C. and allowed to stir vigorously for 24 h. The organic and aqueous fractions were separated, and the aqueous layer was cooled to 0° C. via an ice-water bath. To the viscous solution was added a seed crystal of (S)-methadone hydrochloride to provide a free-flowing slurry. The slurry was allowed to age for 24 h, cooled to 0° C., and the solids were collected via vacuum filtration and dried under reduced pressure at 50° C. for 24 h to provide (S)-6-(dimethylamino)-4,4-diphenyl-3-heptanone hydrochloride ((S)-methadone*HCl) as a white solid (5.20 g, 66% yield). Purity HPLC: 99.6%. Chiral HPLC: one single enantiomer (e.e. >99%) as shown in FIG. 29. HRMS (ESI) m/z: [M+H]⁺ Required C₂₁H₂₇NO 310.2165; Found: 310.2193. ¹H NMR (400 MHz, DMSO_{d6}) δ 10.60 (s, 1H), 7.48-7.24 (m, 10H), 3.10 (d, J=13.9 Hz, 1H), 2.90 (p, J=6.9 Hz, 1H), 2.63 (dd, J=22.7, 4.7 Hz, 6H), 2.47-2.35 (m, 1H), 2.31-2.14 (m, 2H), 0.72 (t, J=7.2 Hz, 3H), 0.48 (d, J=6.5 Hz, 3H) ppm, as shown in FIG. 30. ¹³C NMR (400 MHz, DMSO_{d6}) δ 210.64, 140.53, 140.07, 129.23, 129.06, 128.75, 128.53, 127.61, 64.57, 59.00, 38.78, 38.31, 38.05, 32.40, 14.72, 9.33 ppm, as shown in FIG. 31.

### Example 6: (R)-6-(Dimethylamino)-4,4-Diphenyl-3-Heptanone Hydrochloride ((R)-Methadone*HCl)

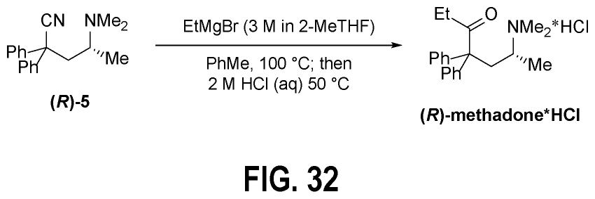

This Example 6 describes a one-step method that was used to prepare (R)-6-(dimethylamino)-4,4-diphenyl-3-heptanone hydrochloride ((R)-methadone*HCl), as shown in FIG. 32. This step was carried out by following literature procedures (see WO 2017/35224 and US2020/277250, incorporated by reference herein in their entireties) with modifications.

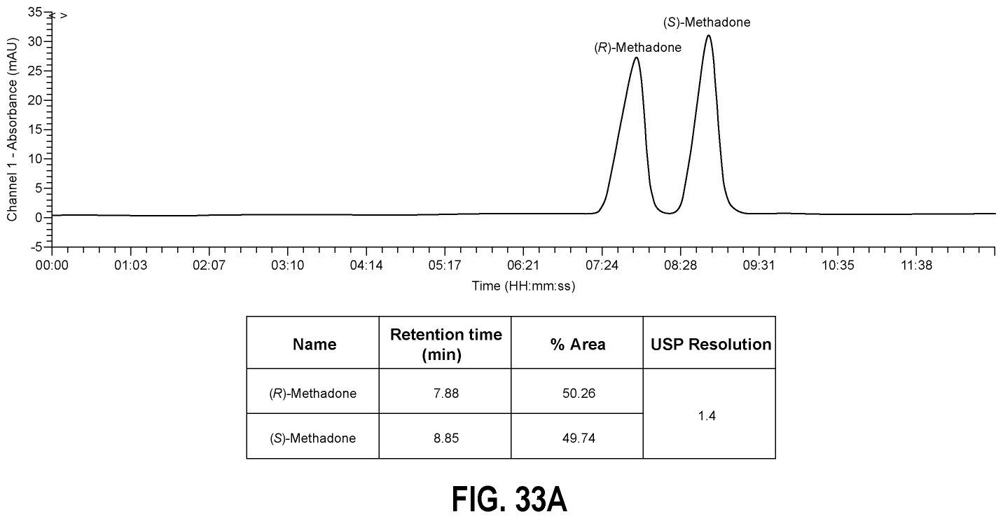

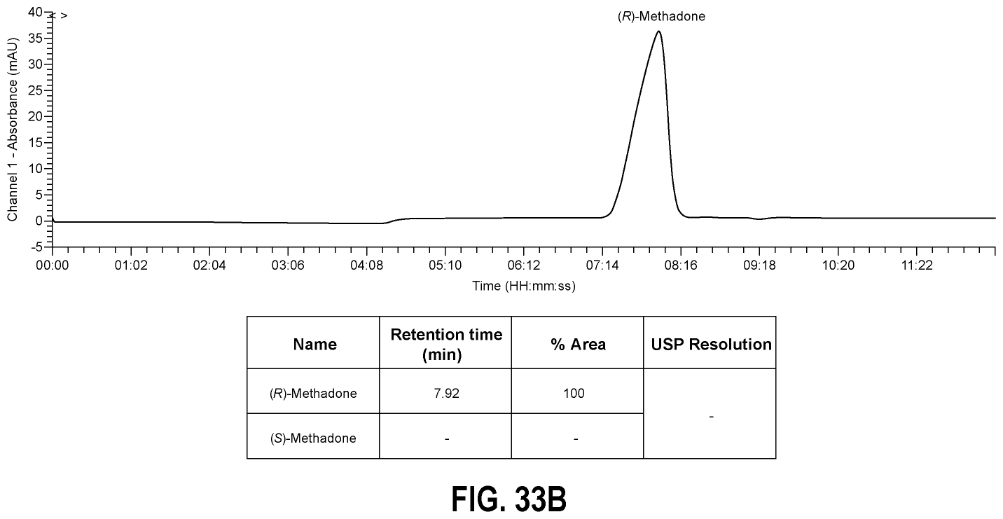

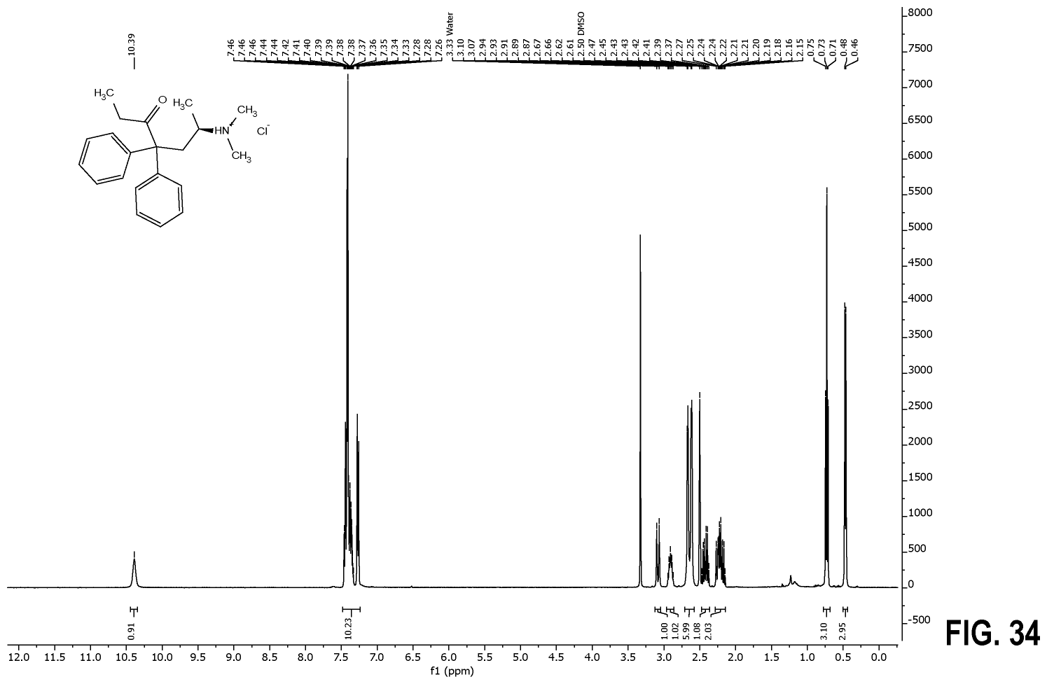

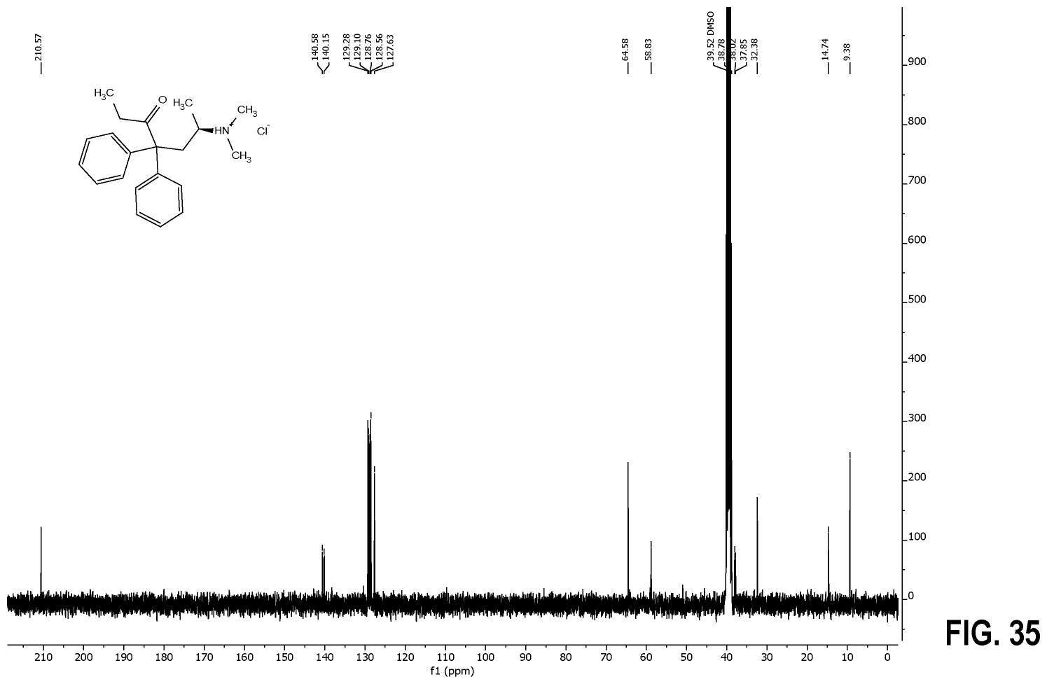

To a 100 mL two-neck round bottom flask equipped with a thermocouple, magnetic stir bar, and N₂ inlet was added (R)-4-(dimethylamino)-2,2-diphenylpentanenitrile ((R)-5) (2.80 g, 10.06 mmol, 1 equiv.). The vessel was purged with N₂ for 30 min, diluted with PhMe (28 mL, 10 vol.), and to the resultant solution was added 3 M EtMgBr in 2-MeTHF (5 mL, 15.09 mmol, 1.5 equiv.) dropwise over 30 min. The reaction mixture was then warmed to 100° C. via a heating mantle and allowed to stir for 24 h. Upon consumption of (R)-5 as determined by the HPLC, the solution was cooled to 0° C., and charged with 2 M aqueous HCl (6 mL, 2 vol.). The biphasic mixture was then warmed to 50° C. and allowed to stir vigorously for 24 h. The organic and aqueous fractions were separated, and the aqueous layer was cooled to 0° C. via an ice-water bath. To the viscous solution was added a seed crystal of (R)-methadone hydrochloride to provide a free-flowing slurry. The slurry was allowed to age for 24 h, cooled to 0° C., and the solids were collected via vacuum filtration and dried under reduced pressure at 50° C. for 24 h to provide (R)-6-(dimethylamino)-4,4-diphenyl-3-heptanone hydrochloride ((R)-methadone*HCl) as a white solid (2.44 g, 70% yield). Purity HPLC: 99.5%. Chiral HPLC: one single enantiomer (e.e. >99%) as shown in FIG. 33. HRMS (ESI) m/z: [M+H]⁺ Required C₂₁H₂₇NO 310.2165; Found: 310.2234. ¹H NMR (400 MHz, DMSO_{d6}) δ 10.39 (s, 1H), 7.48-7.24 (m, 10H), 3.08 (d, J=13.9 Hz, 1H), 2.91 (p, J=7.0 Hz, 1H), 2.64 (dd, J=21.5, 4.5 Hz, 6H), 2.48-2.36 (m, 1H), 2.29-2.13 (m, 2H), 0.73 (t, J=7.2 Hz, 3H), 0.47 (d, J=6.6 Hz, 3H) ppm, as shown in FIG. 34. ¹³C NMR (400 MHz, DMSO_{d6}) δ 210.57, 140.58, 140.15, 129.28, 129.10, 128.76, 128.56, 127.63, 64.58, 58.83, 38.78, 38.02, 37.85, 32.38, 14.74, 9.38 ppm, as shown in FIG. 35.

While the present invention has been disclosed by reference to the details of preferred embodiments of the invention, it is to be understood that the disclosure is intended as an illustrative rather than in a limiting sense, as it is contemplated that modifications will readily occur to those skilled in the art, within the spirit of the invention and the scope of the amended claims.

What is claimed is:

1. A method for preparing (S)-methadone, (R)-methadone or (R,S)-methadone from optically pure ((S)- or (R)-) or racemic N-protected 4-methyl-cyclic sulfamidate or N-protected 2-methylaziridine, the method comprising:
a ring opening reaction of optically pure ((S)- or (R)-) or racemic N-protected 4-methyl-cyclic sulfamidate or N-protected 2-methylaziridine with diphenylacetonitrile in the presence of a base to provide ((S)- or (R)-) or racemic N-protected-dinormethadone nitrile with retention of configuration;
a two-step/one-pot deprotection/reductive amination of the N-protected-dinormethadone nitrile to provide (R)-, (S)-, or (R,S)-methadone nitrile; and
a reaction of the (R)-, (S)-, or (R,S)-methadone nitrile with an organomagnesium halide reagent to form an ethyl-imine intermediate, followed by imine hydrolysis to provide (R)-, (S)-, or (R,S)-methadone.

2. The method of claim 1, wherein the organomagnesium halide reagent is EtMgX, wherein X is selected from Cl, Br, and I.

3. The method of claim 1, wherein the N-protecting group of N-protected 4-methyl-cyclic sulfamidate or N-protected 2-methylaziridine is selected from fluorenylmethyloxycarbonyl (Fmoc), tert-butyloxycarbonyl (Boc), carboxybenzyl (Cbz), acetyl (C(O)CH₃), trifluoroacetyl (C(O)CF₃), benzyl (Bn), triphenylmethyl (CPh₃), and p-toluenesulfonyl (Ts).

4. The method of claim 1, wherein enantiomeric excess (e.e.) is greater than a percentage selected from 90%, 95%, 97%, 99%, 99.5%, and 99.9%.

5. An isolated compound, or a pharmaceutically acceptable salt thereof, prepared by the method of claim 1, the compound having the formula: 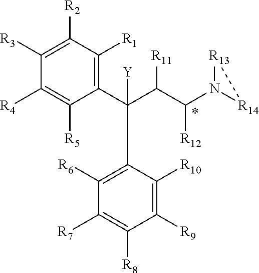
wherein
R₁ to R₁₀ are, independently for each occurrence, hydrogen, deuterium, halogen, alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate;
R₁₁ and R₁₂ are, independently for each occurrence, hydrogen, deuterium, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate; or R₁₁ and R₁₂ are, independently for each occurrence, selected from the group consisting of halogen, alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate;
NR₁₃R₁₄ is optionally cyclized through C₃-C₁₂ cycloalkyl, or heterocyclyl, any of which are optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate;
If NR₁₃R₁₄ is not cyclized R₁₃ and R₁₄ are, independently for each occurrence, hydrogen, deuterium, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate, or R₁₃ and R₁₄ are, independently for each occurrence, selected from the group consisting of alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate;
Y is cyano, —C(O)R₁₅, —C(OH)R₁₅, —C(OR₁₆)R₁₅. R₁₅ and R₁₆ are, independently for each occurrence, hydrogen, deuterium, C₁-C₈ alkyl, C₂-C₈ alkenyl, C₂-C₈ alkynyl, C₃-C₈ cycloalkyl, C₃-C₈ cycloalkenyl, aryl or heterocyclyl, optionally substituted at one or more positions by deuterium, halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate; or R₁₅ and R₁₆ are, independently for each occurrence, selected from the group consisting of alkyl ester, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, nitrate; and
* is a stereocenter selected from (R)-, (S)- or (R,S)-configuration, wherein the stereocenter is in an R configuration, and wherein the compound is present in greater than 90%, 95%, 97%, 99%, 99.5%, or 99.9% enantiomeric excess (e.e) compared to the S isomer, or wherein the stereocenter is in an S configuration, and wherein the compound is present in greater than 90%, 95%, 97%, 99%, 99.5%, or 99.9% enantiomeric excess (e.e) compared to the R isomer.

6. A method for preparing (S)-methadone hydrochloride, (R)-methadone hydrochloride or (R,S)-methadone hydrochloride, the method comprising:
a ring opening reaction of optically pure ((S)- or (R)-) or racemic N-protected 4-methyl-cyclic sulfamidate or N-protected 2-methylaziridine with diphenylacetonitrile in the presence of a base to provide ((S)- or (R)-) or racemic N-protected-dinormethadone nitrile with retention of configuration;
a two-step/one-pot deprotection/reductive amination of the N-protected-dinormethadone nitrile to provide (R)-, (S)-, or (R,S)-methadone nitrile; and
a reaction of the (R)-, (S)-, or (R,S)-methadone nitrile with an organomagnesium halide reagent to form an ethyl-imine intermediate, followed by hydrochloric acid-mediated imine hydrolysis to provide (R)-, (S)-, or (R,S)-methadone hydrochloride.

7. The method of claim 6, wherein the organomagnesium halide reagent is EtMgX, wherein X is selected from Cl, Br, and I.

8. The method of claim 6, wherein the N-protecting group of N-protected 4-methyl-cyclic sulfamidate or N-protected 2-methylaziridine is selected from fluorenylmethyloxycarbonyl (Fmoc), tert-butyloxycarbonyl (Boc), carboxybenzyl (Cbz), acetyl (C(O)CH₃), trifluoroacetyl (C(O)CF₃), benzyl (Bn), triphenylmethyl (CPh₃), and p-toluenesulfonyl (Ts).

9. The method of claim 6, wherein enantiomeric excess (e.e.) is greater than a percentage selected from 90%, 95%, 97%, 99%, 99.5%, and 99.9%.

10. A method for the chromatography free and scalable preparation of a cyclic sulfamidate starting material selected from benzyl (S)-4-methyl-1,2,3-oxathiazolidine-3-carboxylate 2,2-dioxide, (R)-4-methyl-1,2,3-oxathiazolidine-3-carboxylate 2,2-dioxide, and (R,S)-4-methyl-1,2,3-oxathiazolidine-3-carboxylate 2,2-dioxide.

11. A method for preparing (R)-, (S)-, or (R,S)-methadone nitrile, the method comprising:
a ring opening reaction of optically pure ((S)- or (R)-) or racemic N-protected 4-methyl-cyclic sulfamidate or N-protected 2-methylaziridine with diphenylacetonitrile in the presence of a base to provide ((S)- or (R)-) or racemic N-protected-dinormethadone nitrile with retention of configuration;
a two-step/one-pot deprotection/reductive amination of the N-protected-dinormethadone nitrile to provide (R)-, (S)-, or (R,S)-methadone nitrile.
